(19) United States
(12) Patent Application Publication
(10) Pub. No.: US 2021/0403868 A1
(43) Pub. Date: Dec. 30, 2021

(54) DEVICE FOR CELL CULTURE AND CELL CULTURING METHOD

(71) Applicant: DEXERIALS CORPORATION, Tokyo (JP)

(72) Inventors: Hirokazu ODAGIRI, Tokyo (JP); Keiji Honjo, Tokyo (JP); Misao Konishi, Tokyo (JP); Koji Eto, Chiba (JP); Naoya Takayama, Chiba (JP)

(73) Assignee: DEXERIALS CORPORATION, Tokyo (JP)

(21) Appl. No.: 17/304,687

(22) Filed: Jun. 24, 2021

(30) Foreign Application Priority Data
Jun. 29, 2020 (JP) 2020-111779
Jun. 15, 2021 (JP) 2021-099354

(51) Int. Cl. C12N 5/0789

(52) U.S. Cl. CPC C12N 5/0647; C12N 2535/00; C12N 2533/30

(57) ABSTRACT

Provided is a device for cell culture, the device including: a base material including a culture section used for culturing hematopoietic stem cells or hematopoietic progenitor cells, or both the hematopoietic stem cells and the hematopoietic progenitor cells, wherein the culture section includes a plurality of pores, and wherein a Young's modulus of the culture section measured according to JIS K 7161-1 and JIS K 7161-2 is at least 3 GPa.

### CROSS-REFERENCE TO RELATED APPLICATIONS

This application claims priority to Japanese application No. 2020-111779, filed on Jun. 29, 2020 and Japanese application No. 2021-099354, filed on Jun. 15, 2021 and incorporated herein by reference.

### BACKGROUND OF THE INVENTION

### Field of the Invention

The present invention relates to a device for cell culture and a cell culturing method.

### Description of the Related Art

Hematopoietic stem cells (HSC) are cells that have both multipotency to differentiate into various hematopoietic cells such as white blood cells (e.g., neutrophils, eosinophils, basophils, lymphocytes, monocytes, and macrophages), red blood cells, platelets, mast cells, and dendritic cells, and self-replication ability to replicate themselves while maintaining the multipotency. Hematopoietic stem cells are known to follow a differentiation process of differentiating into hematopoietic progenitor cells (also referred to as “multipotent hematopoietic progenitor cells”) first and then differentiating into various hematopoietic cells via various progenitor cells.

Hence, hematopoietic stem cells and hematopoietic progenitor cells are both important cells that may be applicable to treatment of blood cancers such as leukemia, malignant lymphoma, and multiple myeloma.

Hematopoietic stem cells and hematopoietic progenitor cells have a cell size of about from 10 micrometers through 15 micrometers, and are generally said to exist in a special microenvironment called Niche in the bone marrow and maintain the balance among retention in stationary phase, self-replication, and differentiation via crosstalks between hematopoietic stem cells or between hematopoietic progenitor cells, and via, for example, humoral factors and intercellular adhesion factors from the surrounding environment. The physical space of the microenvironment is the cancellous bone in the bone marrow. Therefore, attempts have been made recently to imitate the structure of the cancellous bone as a scaffold for proliferating hematopoietic stem cells and hematopoietic progenitor cells in vitro.

For example, a proposed method cultures hematopoietic progenitor cells in vitro using a porous solid matrix (see Japanese Patent Application Laid-Open (JP-A) No. 2001-517428). The porous solid matrix has an open cell structure in which pores are reticulated and joined. However, it is industrially challenging and costly to produce such porous solid matrices having a unitary microstructure, and it is difficult to mass-produce porous solid matrices.

Metal coating over the porous solid matrix is also proposed for structural reinforcement and improvement of adhesiveness of cells to the solid matrix. However, it is also challenging and yield-reducing to coat also the pores in the porous body uniformly. Also in this respect, high costs arise as a problem.

Another proposed method cultures undifferentiated cells such as human ES cells using a culture carrier formed of a ceramic or glass base material in whose surface a plurality of concaves formed of a porous body are arranged in a matrix (see JP-A No. 2008-306987). ES cells lose their undifferentiated state when the colony size becomes a certain level or greater. By controlling the colony size by the concaves, the proposed culture carrier can obtain an aggregation of cells that have proliferated remaining undifferentiated.

However, the proposed culture carrier has too small a depth-to-diameter ratio (hereinafter, may be referred to as “aspect ratio”) to be applied as a niche (microenvironment) for hematopoietic stem cells and hematopoietic progenitor cells, and is problematic in that the cells are clustered sparsely. There is another problem that, for example, ceramics are costly as the material of the culture carrier and unsuitable for mass culturing.

Hence, a device for cell culture that imitates the structure of the cancellous bone as a scaffold for proliferating hematopoietic stem cells or hematopoietic progenitor cells, or both the hematopoietic stem cells and the hematopoietic progenitor cells in vitro, can be produced easily at low costs, and can efficiently proliferate hematopoietic stem cells or hematopoietic progenitor cells, or both the hematopoietic stem cells and the hematopoietic progenitor cells while maintaining the self-replication ability and the multipotency thereof, and a cell culturing method have not been provided yet, and provision thereof is currently strongly demanded.

### SUMMARY OF THE INVENTION

The present invention aims for solving the various problems in the related art described above and achieving an object described below. That is, the present invention has an object to provide a device for cell culture that can be produced easily at low costs and can efficiently proliferate hematopoietic stem cells or hematopoietic progenitor cells, or both the hematopoietic stem cells and the hematopoietic progenitor cells in vitro while maintaining the self-replication ability and the multipotency thereof, and a cell culturing method that can efficiently proliferate hematopoietic stem cells or hematopoietic progenitor cells, or both the hematopoietic stem cells and the hematopoietic progenitor cells in vitro while maintaining the self-replication ability and the multipotency thereof.

Aspects of the present invention are as follows.

<1> A device for cell culture, the device including:

a base material including a culture section used for culturing hematopoietic stem cells or hematopoietic progenitor cells, or both the hematopoietic stem cells and the hematopoietic progenitor cells,

wherein the culture section includes a plurality of pores, and

wherein a Young's modulus of the culture section measured according to JIS K 7161-1 and JIS K 7161-2 is at least 3 GPa.

<2> The device for cell culture according to <1>,

wherein the culture section is formed of polystyrene.

<3> The device for cell culture according to <1> or <2>,

wherein a ratio [H/La] of an average depth (H) of the pores to an average length (La) of openings of the pores is from 1.0 through 2.0.

<4> The device for cell culture according to any one of <1> to <3>,

wherein an average length of openings of the pores is from 30 micrometers through 80 micrometers.

<5> The device for cell culture according to any one of <1> to <4>,

wherein an average depth of the pores is from 30 micrometers through 160 micrometers.

<6> A cell culturing method, including:

culturing hematopoietic stem cells or hematopoietic progenitor cells, or both the hematopoietic stem cells and the hematopoietic progenitor cells using the device for cell culture according to any one of <1> to <5>.

The present invention can solve the various problems in the related art described above, achieve the object described above, and can provide a device for cell culture that can be produced easily at low costs and can efficiently proliferate hematopoietic stem cells or hematopoietic progenitor cells, or both the hematopoietic stem cells and the hematopoietic progenitor cells in vitro while maintaining the self-replication ability and the multipotency thereof, and a cell culturing method that can efficiently proliferate hematopoietic stem cells or hematopoietic progenitor cells, or both the hematopoietic stem cells and the hematopoietic progenitor cells in vitro while maintaining the self-replication ability and the multipotency thereof.

### BRIEF DESCRIPTION OF THE DRAWINGS

FIG. 1 is a schematic view (perspective view) illustrating an example of a state in which a device 20 for cell culture is placed in a well 1 of a known culture container (96-well plate);

FIG. 2A is a schematic view (perspective view) illustrating an example of a device 20 for cell culture including a culture section 21 and a handle 22;

FIG. 2B is a cross-sectional view of the device 20 for cell culture of FIG. 2A taken along a line A-A;

FIG. 3 is an enlarged top view of a part of a cell seeding surface 21a of a culture section 21 of a device 20 for cell culture including a plurality of pores 30, where X and Y each independently represent the length of a side of an opening 31 of a pore 30 and p represents pitch;

FIG. 4 is a cross-sectional view of the culture section 21 of FIG. 3 including the plurality of pores 30 taken along a line B-B, where X represents the length of a side of an opening 31 of a pore 30, p represents pitch, and h represents the depth of a pore 30;

FIG. 5A is a schematic view illustrating an example of a method for forming pores in a base material of a device for cell culture, illustrating an example of a step of aligning a master plate 40 engraved with an inverse shape of a plurality of pores present in a surface of a culture section of the device for cell culture, with a resin film 41 serving as the base material;

FIG. 5B is a schematic view illustrating an example of a method for forming pores in a base material of a device for cell culture, illustrating an example of a step of pressing the master plate 40 against the resin film 41 after the step illustrated in FIG. 5A;

FIG. 5C is a schematic view illustrating an example of a method for forming pores in a base material of a device for cell culture, illustrating an example of a step of releasing the resin film 41 from the master plate 40 after the step illustrated in FIG. 5B to obtain the base material, which is the resin film 41 to which the shape of the master plate 40 is transferred;

FIG. 6 is a schematic view (top view) illustrating an example of a step of punching out a device for cell culture including a culture section 21, a handle 22, and a joint 23 from a resin film 41;

FIG. 7 is a graph indicating the result of analyzing cell surface markers of hematopoietic stem cells among cells that are cultured using devices for cell culture of Example 1 and Comparative Example 1 or among cells cultured as control, where the vertical axis represents the number of cells of a hematopoietic stem cell fraction that is positive with CD34, positive with CD90, and negative with CD45RA (hereinafter, may be referred to as [CD34+, CD90+, and CD45RA− cells]);

FIG. 8 is a graph indicating the result of analyzing cell surface markers of hematopoietic stem cells among cells cultured using devices for cell culture of Examples 1 to 3, where the vertical axis represents the number of cells of a hematopoietic stem cell fraction [CD34+, CD90+, and CD45RA− cells];

FIG. 9A illustrates an example of a phase-contrast microscope image of umbilical cord blood-derived CD34-positive cells, observed after cultured for seven days using a device for cell culture of Example 1, where the scale bar represents 100 micrometers;

FIG. 9B illustrates an example of a phase-contrast microscope image of umbilical cord blood-derived CD34-positive cells, observed after cultured for seven days using a device for cell culture of Example 2, where the scale bar represents 50 micrometers;

FIG. 9C illustrates an example of a phase-contrast microscope image of umbilical cord blood-derived CD34-positive cells, observed after cultured for seven days using a device for cell culture of Example 3, where the scale bar represents 200 micrometers;

FIG. 10 is a graph indicating the result of analyzing cell surface markers of hematopoietic stem cells among cells cultured using devices for cell culture of Example 1 and Example 6, where the vertical axis represents the number of cells of a hematopoietic stem cell fraction [CD34+, CD90+, and CD45RA− cells];

FIG. 11 is a graph indicating the result of analyzing cell surface markers of hematopoietic stem cells among cells cultured using devices for cell culture of Example 1, Example 2, Example 4, and Example 5, where the vertical axis represents the number of cells of a hematopoietic stem cell fraction [CD34+. CD90+, and CD45RA− cells];

FIG. 12 is a graph indicating the result of analyzing cell surface markers of hematopoietic stem cells among cells cultured using devices for cell culture of Example 1, Example 2, Example 4, Example 5, and Example 6, where the vertical axis represents the number of cells of a fraction including hematopoietic stem cells and hematopoietic progenitor cells [CD34+ cells];

FIG. 13 is a graph indicating the result of a methyl cellulose colony assay of cells cultured using devices for cell culture of Examples 1 to 3, where the vertical axis represents the number of colonies, where in the bottom-up order, the bars represent the number of colonies of granulocytic lineage and monocytic lineage progenitor cells by dark gray, the number of colonies of burst-forming unit-erythroid by oblique lines, and the number of mixed colonies in which blood cells of a plurality of lineages are mixed by pale gray;

FIG. 14A is a schematic diagram illustrating an example of a method for calculating an average length (La1) when the shape formed by the outer boundary of an opening (top view) of a pore is a polygon; and

FIG. 14B is a schematic diagram illustrating an example of a method for calculating an average length (La2) when the shape formed by the outer boundary of an opening (top view) of a pore is not a polygon.

### DESCRIPTION OF THE EMBODIMENTS

### (Device for Cell Culture)

A device for cell culture of the present invention includes a base material including a culture section used for culturing hematopoietic stem cells or hematopoietic progenitor cells, or both the hematopoietic stem cells and the hematopoietic progenitor cells (hereinafter, may be abbreviated simply as “cells”), and further includes other components as needed.

The device for cell culture may be used alone for culturing hematopoietic stem cells or hematopoietic progenitor cells, or both the hematopoietic stem cells and the hematopoietic progenitor cells, or may be used as an insert in a known culture container.

When the device for cell culture is used alone for culturing hematopoietic stem cells or hematopoietic progenitor cells, or both the hematopoietic stem cells and the hematopoietic progenitor cells, the shape of the device is not particularly limited and may be appropriately selected depending on the intended purpose.

Examples of the shape of the device include the same shape as a known culture container.

In the present invention, an “insert” means a member that is used being stacked in a well of a known culture container or in a dish. When the device for cell culture is used as the insert, the device for cell culture may or may not be placed in contact with the bottom of a well of the known culture container or the bottom of the dish so long as hematopoietic stem cells or hematopoietic progenitor cells, or both the hematopoietic stem cells and the hematopoietic progenitor cells can be cultured in the culture section.

### <Base Material>

The base material includes a culture section, and further includes other members as needed.

The shape of the base material is not particularly limited and may be appropriately selected depending on the intended purpose so long as a plurality of pores can be provided in the culture section of the base material. Examples of the shape of the base material include a sheet shape, a film shape, a plate shape, and a board shape.

The base material may have a single-layer structure or a multilayer structure.

The average thickness of the base material is not particularly limited, may be appropriately selected depending on, for example, the depth of the pores, and is preferably from 50 micrometers through 300 micrometers and more preferably from 100 micrometers through 200 micrometers. An average thickness of the base material of 50 micrometers or greater is preferable in terms of warpage and bending of the base material. An average thickness of the base material of 300 micrometers or less is preferable in terms of punching processing.

The average thickness of the base material is an average calculated from thickness measurements obtained at arbitrary ten positions of the base material using a micrometer MDC-25MX (No. 293-230-30, available from Mitutoyo Corporation).

### <<Culture Section>>

The culture section is a member used for culturing hematopoietic stem cells or hematopoietic progenitor cells, or both the hematopoietic stem cells and the hematopoietic progenitor cells.

The culture section includes a plurality of pores, and further includes other components as needed.

The shape of the culture section is not particularly limited and may be appropriately selected depending on the intended purpose. Examples of the shape of the culture section include: circles such as perfect circles (true circles) and ellipses; polygons such as triangles, quadrangles, hexagons, and octagons that may have different lengths on the respective sides; and combinations of these shapes. When the device for cell culture is used as the insert, the shape of the culture section may be appropriately selected to suit to the shape of the culture container to which the insert is applied.

The Young's modulus of the culture section measured according to JIS K 7161-1 and JIS K 7161-2 is at least 3 GPa. When the Young's modulus of the culture section is less than 3 GPa, the proliferation efficiency of hematopoietic stem cells or hematopoietic progenitor cells, or both the hematopoietic stem cells and the hematopoietic progenitor cells in vitro is poor.

It is important that the Young's modulus of the culture section be at least 3 GPa, in order to put the culture section under conditions very similar to the environment in the bone marrow, i.e., to make the culture section as hard as the cancellous bone. Hence, the upper limit of the Young's modulus of the culture section is not particularly limited and may be appropriately selected depending on the intended purpose.

The material of the culture section having a Young's modulus of at least 3 GPa is not particularly limited and may be appropriately selected from resin materials commonly used. Examples of the resin materials include thermoplastic resins that deform or extend in response to heat, and ultraviolet-curable resins that cure from liquids to solids in response to light energy of ultraviolet rays.

Examples of the thermoplastic resins include polystyrene, polycarbonate, polyamide, polyvinyl alcohol, polylactic acid, and copolymers of polylactic acid and polyglycolic acid. One of these thermoplastic resins may be used alone or two or more of these thermoplastic resins may be used in combination. Among these thermoplastic resins, polystyrene is preferable.

Examples of the ultraviolet-curable resins include acrylate-based and urethane acrylate-based resins. One of these ultraviolet-curable resins may be used alone or two or more of these ultraviolet-curable resins may be used in combination.

### Pores

It is preferable that the pores of the culture section be blind holes (pores) communicating to the outside of the culture section at one end in the thickness direction of the culture section (or the thickness direction of the base material), because this makes it easy to make the pores uniform in depth.

The blind holes may be formed as concaves in or convexes from the surface of the base material. However, it is preferable that the blind holes be formed as concaves because it is easier to form concaves.

In the culture section, it is preferable that the blind holes be provided only in one surface of the culture section. In this case, it is preferable to use the surface of the culture section provided with the blind holes therein as a surface to which hematopoietic stem cells or hematopoietic progenitor cells, or both the hematopoietic stem cells and the hematopoietic progenitor cells are seeded (hereinafter, may be referred to as “cell seeding surface”).

Through entry into the pores, hematopoietic stem cells or hematopoietic progenitor cells, or both the hematopoietic stem cells and the hematopoietic progenitor cells come to have an appropriate cell density in the pores, and, because of crosstalks between the cells (e.g., paracrine factors and autocrine factors), are efficiently proliferated in vitro with the self-replication ability and the multipotency thereof maintained. Hematopoietic stem cells or hematopoietic progenitor cells, or both the hematopoietic stem cells and the hematopoietic progenitor cells that have entered the pores not only two-dimensionally proliferate over the cell seeding surface in the pores, but also three-dimensionally proliferate in the depth direction of the pores. This is advantageous because the environment in the pores is more similar to the conditions of the cancellous bone.

Hence, it is preferable to culture hematopoietic stem cells or hematopoietic progenitor cells, or both the hematopoietic stem cells and the hematopoietic progenitor cells in the pores.

The pattern of the plurality of pores when the culture section is seen from above is not particularly limited and may be appropriately selected depending on the intended purpose. Examples of the pattern include linear patterns, curved patterns, broken line patterns, concentric patterns, grid patterns, honeycomb patterns, and combinations of these patterns.

The pattern of the plurality of pores may be regular or irregular. However, a regular pattern is preferable because it is easier to produce the device for cell culture.

The plurality of pores may be provided at a part of the culture section or may be provided all over the culture section. However, it is preferable that the plurality of pores be provided all over the culture section because the proliferation efficiency of hematopoietic stem cells or hematopoietic progenitor cells, or both the hematopoietic stem cells and the hematopoietic progenitor cells is good.

When the plurality of pores are provided at a part of the culture section, the positions and the size of the plurality of pores in the culture section are not particularly limited and may be appropriately selected depending on the intended purpose.

The number of pores in the culture section is not particularly limited and may be appropriately selected depending on, for example, the size of the culture section or the device for cell culture.

The shape formed by the outer boundary of the opening of each pore when the culture section is seen from above is not particularly limited and may be appropriately selected depending on the intended purpose. Examples of the shape include: circles such as perfect circles (true circles) and ellipses; polygons such as triangles, quadrangles, hexagons, and octagons that may have different lengths on the respective sides; and combinations of these shapes. Among these shapes, the shape formed by the outer boundary of the opening of each pore is preferably a perfect circle or a regular polygon having the same length on the respective sides because it is easy to produce the device for cell culture.

All of the plurality of pores may be the same or different in the shape formed by the outer boundary of the opening. It is preferable that all of the plurality of pores be the same in the shape formed by the outer boundary of the opening because it is easy to produce the device for cell culture.

The opening area of the opening of each pore when the culture section is seen from above is not particularly limited, may be appropriately selected depending on the intended purpose, and is preferably from 2.25×10⁻⁴ mm² through 0.01 mm², more preferably from 0.0009 mm² through 0.0064 mm², and yet more preferably from 0.0016 mm² through 0.0036 mm². The opening area of the opening of each pore when the culture section is seen from above is the area of the figure formed by the outer boundary of the opening of each pore.

Opening areas of the opening of each pore in cross-sections taken in the horizontal direction of the opening when the culture section is seen from above are not particularly limited, may be appropriately selected depending on the intended purpose, and may or may not change from the bottom to the opening. When the opening areas change from the bottom of the pore to the opening, the pore may have a shape having a gradually increasing opening area.

The average length (La) of the openings of the pores is not particularly limited, may be appropriately selected depending on, for example, the number of the pores and the average pitch between the openings, and is preferably from 15 micrometers through 100 micrometers, more preferably from 30 micrometers through 80 micrometers, and particularly preferably from 40 micrometers through 60 micrometers. Because hematopoietic stem cells and hematopoietic progenitor cells have a diameter of about from 10 micrometers through 15 micrometers, an average length (La) of the openings of 15 micrometers or greater is preferable because hematopoietic stem cells or hematopoietic progenitor cells, or both the hematopoietic stem cells and the hematopoietic progenitor cells can easily enter the pores. An average length (La) of the openings of 100 micrometers or less is preferable because hematopoietic stem cells or hematopoietic progenitor cells, or both the hematopoietic stem cells and the hematopoietic progenitor cells that have entered the pores have an appropriate density and can be efficiently proliferated in vitro with the self-replication ability and the multipotency thereof maintained.

In the present invention, the “average length (La)” means one of “average length (La1)”, “average length (La2)”, and “average length (La3)” described below depending on the shape formed by the outer boundary of the opening of the pore.

In the present invention, when the shape formed by the outer boundary of the opening of a pore is a polygon, the average length (La1) of the openings of the pores is calculated as follows.

After the length of each side of the polygon formed by the outer boundary of the opening of one pore arbitrarily selected is measured, an average (Sa) of the lengths of all the sides is calculated. Such an average (Sa) is calculated for ten pores arbitrarily selected, as an average (Sa₁), an average (Sa₂), an average (Sa₃), an average (Sa₄), an average (Sa₅), an average (Sa₆), an average (Sa₇), an average (Sa₈), an average (Sa₉), and an average (Sa₁₀). Next, an average of the averages (Sa₁) to (Sa₁₀) is calculated as “average length (La1)”.

When the number of pores in the device for cell culture is a number (n) less than ten, an average of the averages (Sa₁) to (Saₙ) is calculated as “average length (La1)”.

The length of each side of the shape formed by the outer boundary of the opening can be measured with a field-emission scanning electron microscope S-4700 (available from Hitachi High-Technologies Corporation).

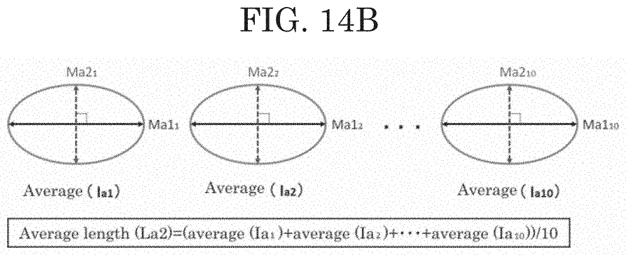

The average length (La1) will be more specifically described with reference to FIG. 14A. For example, consider a case where the shape formed by the outer boundary of the opening of the pore is a quadrangle as illustrated in FIG. 14A. In this case, after the lengths of the side a₁, the side b₁, the side c₁, and the side d₁ of the quadrangle formed by the outer boundary of the opening of one pore arbitrarily selected are measured, the average (Sa₁) of the lengths of all the sides is calculated according to the formula (1-1) below. Likewise, averages (Sa₂) to (Sa₁₀) are calculated according to the formulae (1-2) to (1-10) below for any other nine pores arbitrarily selected. Next, an average of the averages (Sa₁) to (Sa₁₀) is calculated according to the formula (1-11) below. In this way, the “average length (La1)” can be calculated.

Average(Sa₁)=(a₁+b₁+c₁+d₁)/4 Formula(1-1)

Average(Sa₂)=(a₂+b₂+c₂+d₂)/4 Formula(1-2)

Average(Sa₃)=(a₃+b₃+c₃+d₄)/4 Formula(1-3)

Average(Sa₄)=(a₄+b₄+c₄+d₄)/4 Formula(1-4)

Average(Sa₅)=(a₅+b₅+c₅+d₅)/4 Formula(1-5)

Average(Sa₆)=(a₆+b₆+c₆+d₆)/4 Formula(1-6)

Average(Sa₇)=(a₇+b₇+c₇+d₇)/4 Formula(1-7)

Average(Sa₈)=(a₈+b₈+c₈+d₈)/4 Formula(1-8)

Average(Sa₉)=(a₉+b₉+c₉+d₉)/4 Formula(1-9)

Average(Sa₁₀)=(a₁₀+b₁₀+c₁₀+d₁₀)/4 Formula(1-10)

Average length(La1)=[(Sa₁)+(Sa₂)+(Sa₃)+(Sa₄)+(Sa₅)+(Sa₆)+(Sa₇)+(Sa₈)+(Sa₉)+(Sa₁₀)]/10 Formula(1-11)

In the present invention, when the shape formed by the outer boundary of the opening of a pore is not a polygon, the average length (La2) of the openings of the pores is calculated as follows.

After the maximum length (Ma1) of the shape formed by the outer boundary of the opening of one pore arbitrarily selected and the maximum length (Ma2) in the direction orthogonal to the maximum length (Ma1) are measured, an average (Ia) of the maximum length (Ma1) and the maximum length (Ma2) is calculated. Such an average is calculated for ten pores arbitrarily selected, as an average (Ia₁), an average (Ia₂), an average (Ia₃), an average (Ia₄), an average (Ia₅), an average (Ia₆), an average (Ia₇), an average (Ia₈), an average (Ia₉), and an average (Ia₁₀). Next, an average of the averages (Ia₁) to (Ia₁₀) is calculated as “average length (La2)”.

When the number of pores in the device for cell culture is a number (n) less than ten, an average of averages (Ia₁) to (Iaₙ) is calculated as “average length (La2)”.

The maximum length (Ma1) of the shape formed by the outer boundary of the opening and the maximum length (Ma2) in the direction orthogonal to the maximum length (Ma1) can be measured with a field-emission scanning electron microscope S-4700 (available from Hitachi High-Technologies Corporation).

The average length (La2) will be described more specifically with reference to FIG. 14B. For example, consider a case where the shape formed by the outer boundary of the opening of a pore is an ellipse as illustrated in FIG. 14B. In this case, after the maximum length (Ma1₁) (represented by a solid line in FIG. 14B) of the ellipse formed by the outer boundary of the opening of one pore arbitrarily selected and the maximum length (Ma2₁) (represented by a broken line in FIG. 14B) in the direction orthogonal to the maximum length (Ma1₁) are measured, an average (Ia₁) of the maximum length (Ma1₁) and the maximum length (Ma2₁) is calculated according to the formula (2-1) below. Likewise, averages (Ia₂) to (Ia₁₀) are calculated according to the formulae (2-2) to (2-10) below for any other arbitrary nine pores. Next, an average of the averages (Ia₁) to (Ia₁₀) is calculated according to the formula (2-11) below. In this way, the “average length (La2)” can be calculated.

Average(Ia₁)=(Ma1₁+Ma2₁)/2 Formula(2-1)

Average(Ia₂)=(Ma1₂+Ma2₂)/2 Formula(2-2)

Average(Ia₃)=(Ma1₃+Ma2₃)/2 Formula(2-3)

Average(Ia₄)=(Ma1₄+Ma2₄)/2 Formula(2-4)

Average(Ia₅)=(Ma1₅+Ma2₅)/2 Formula(2-5)

Average(Ia₆)=(Ma1₆+Ma2₆)/2 Formula(2-6)

Average(Ia₇)=(Ma1₇+Ma2₇)/2 Formula(2-7)

Average(Ia₈)=(Ma1₈+Ma2₈)/2 Formula(2-8)

Average(Ia₉)=(Ma1₉+Ma2₉)/2 Formula(2-9)

Average(Ia₁₀)=(Ma1₁₀+Ma2₁₀)/2 Formula(2-10)

Average length(La2)=[(Ia₁)+(Ia₂)+(Ia₃)+(Ia₄)+(Ia₅)+(Ia₆)+(Ia₇)+(Ia₈)+(Ia₉)+(Ia₁₀)]/10Formula(2-11)

The average length (La3) when polygons and shapes that are not polygons are present simultaneously in the device for cell culture as the shapes formed by the outer boundaries of the openings of the pores is calculated according to the formula (3) below.

Average length(La3)=[average length(La1)+average length(La2)]/2 Formula(3)

The shape of a cross-section of the pore in the depth direction (the thickness direction of the base material) is not particularly limited and may be appropriately selected depending on the intended purpose. Examples of the shape include: shapes (e.g., semicircles) obtained by partially chipping the circumference of circles such as perfect circles (true circles) and ellipses; polygons such as triangles (e.g., a V letter shape), quadrangles (e.g., rectangles, bidirectionally tapered shapes, and unidirectionally tapered shapes), hexagons, and octagons that may have different lengths on the respective sides; and combinations of these shapes (e.g., a shape, of which sides extending in the depth direction in the cross-section of the pore taken along the depth direction (i.e., sides perpendicular to the opening or the bottom) are straight lines and which is U letter-shaped at only the bottom of the pore). All of the plurality of pores may be the same or different in the shape of the cross-section taken along the depth direction.

The average depth (H) of the pores is not particularly limited, may be appropriately selected depending on, for example, the thickness of the base material, and is preferably from 15 micrometers through 160 micrometers, more preferably from 30 micrometers through 160 micrometers, and yet more preferably from 40 micrometers through 100 micrometers. As described above, hematopoietic stem cells or hematopoietic progenitor cells have a diameter of about from 10 micrometers through 15 micrometers. Therefore, an average depth (H) of the pores of 15 micrometers or greater is preferable because hematopoietic stem cells or hematopoietic progenitor cells, or both the hematopoietic stem cells and the hematopoietic progenitor cells can enter the pores. An average depth (H) of the pores of 160 micrometers or less is preferable because hematopoietic stem cells or hematopoietic progenitor cells, or both the hematopoietic stem cells and the hematopoietic progenitor cells that have entered the pores have an appropriate density and can be efficiently proliferated in vitro with the self-replication ability and the multipotency thereof maintained.

In the present invention, the depth (or may be referred to as “length” or “height”) of the pore from a surface of the base material serving as a reference surface to the bottom of the pore in the thickness direction of the base material of the culture section (or in the direction perpendicular to the surface of the base material serving as the reference surface) is defined as “depth (h) of the pore”. When the bottom of the pore is not flat, the depth at the deepest portion is defined as “depth (h) of the pore”. An average of “depths (h) of the pore” measured at ten pores arbitrarily selected is defined as “average depth (H)”.

The depth (h) of the pore can be measured with a field-emission scanning electron microscope S-4700 (available from Hitachi High-Technologies Corporation).

The ratio [H/La] of the average depth (H) of the pores to the average length (La) of the openings (hereinafter, may be referred to as “aspect ratio”) is not particularly limited, may be appropriately selected depending on the intended purpose, and is preferably 0.5 or greater, more preferably 1.0 or greater, and particularly preferably 1.1 or greater. An aspect ratio of 0.5 or greater is preferable because hematopoietic stem cells or hematopoietic progenitor cells, or both the hematopoietic stem cells and the hematopoietic progenitor cells can easily enter the pores and hematopoietic stem cells or hematopoietic progenitor cells, or both the hematopoietic stem cells and the hematopoietic progenitor cells that have entered the pores have an appropriate density and can be efficiently proliferated in vitro with the self-replication ability and the multipotency maintained. The lower limit of the aspect ratio is important in terms of putting the culture section under conditions very similar to the environment in the bone marrow. Hence, the upper limit of the aspect ratio is not particularly limited and may be appropriately selected depending on the intended purpose, but is preferably 2.0 or less because oxygen and nutrients can be sufficiently supplied to hematopoietic stem cells or hematopoietic progenitor cells, or both the hematopoietic stem cells and the hematopoietic progenitor cells that have entered the pores.

All of the plurality of pores may be the same or different in the aspect ratio.

When the pattern of the plurality of pores is a regular pattern and the shape formed by the outer boundaries of the openings of the pores is a shape having a center (e.g., a true circle and a square), the average pitch (P) between the openings of the plurality of pores is not particularly limited, may be appropriately selected depending on the intended purpose, and preferably satisfies the formula (4-1) below and more preferably satisfies the formula (4-2) below. When the average pitch (P) is greater than the range of the formula (4-1) below, hematopoietic stem cells or hematopoietic progenitor cells, or both the hematopoietic stem cells and the hematopoietic progenitor cells cannot enter the plurality of pores efficiently, and may not be proliferated efficiently.

P≤2La Formula(4-1)

1La<P≤2La Formula(4-2)

In the formula (4-1) and the formula (4-2), “P” represents the average pitch and “La” represents the average length of the openings of the pores.

In the present invention, when the pattern and the shape of the pores are as described above, the minimum center-to-center distance between the center of the shape formed by the outer boundary of the opening of one pore arbitrarily selected and the center of the shape formed by the outer boundary of the opening of another pore adjoining the one pore arbitrarily selected is defined as “pitch (p)”. An average of the minimum center-to-center distances measured for ten pores arbitrarily selected is defined as “average pitch (P)”.

The pitch (p) can be measured by observation of the surface of the culture section with a field-emission scanning electron microscope S-4700 (available from Hitachi High-Technologies Corporation).

When the pattern of the plurality of pores is an irregular pattern or the shape formed by the outer boundaries of the openings of the pores is a shape having no center, the minimum distance between the outer boundary of the opening of one pore arbitrarily selected and the outer boundary of the opening of another pore adjoining the one pore arbitrarily selected is defined as “pitch (p)”. An average of the minimum distances measured for ten pores arbitrarily selected is defined as “average pitch (P)”.

The pore density of the plurality of pores in the culture section is not particularly limited, may be appropriately selected depending on, for example, the average length (La), the average depth (H), and the average pitch (P) of the openings of the pores, and is preferably from 2,000 pores/cm² through 160,000 pores/cm² and more preferably from 20,000 pores/cm² through 63,000 pores/cm². When the pore density is less than 2,000 pores/cm² or greater than 160,000 pores/cm², there may be a case where hematopoietic stem cells or hematopoietic progenitor cells, or both the hematopoietic stem cells and the hematopoietic progenitor cells cannot be proliferated efficiently.

The shape formed by the outer boundary of the bottom of the pore when the culture section is seen from above is not particularly limited and may be appropriately selected depending on the intended purpose. Examples of the shape include the shapes raised as examples of the shape formed by the outer boundary of the opening of the pore. It is preferable that the shape formed by the outer boundary of the bottom of the pore when the culture section is seen from above be the same as the shape formed by the outer boundary of the opening of the pore, because it is easy to produce the device for cell culture.

The shape of the bottom in a cross-section of the pore taken along the depth direction is not particularly limited and may be appropriately selected depending on the intended purpose. Examples of the shape include a flat shape, an approximately flat shape, an arc shape, an approximately arc shape, and combinations of these shapes. All of the plurality of pores may be the same or different in the shape of the bottom.

The average length (Lb) of the bottoms of the pores is not particularly limited, may be appropriately selected depending on the intended purpose, and is preferably from 15 micrometers through 100 micrometers, more preferably from 30 micrometers through 80 micrometers, and yet more preferably from 40 micrometers through 60 micrometers. An average length (Lb) of the bottoms of 15 micrometers or greater is preferable because hematopoietic stem cells or hematopoietic progenitor cells, or both the hematopoietic stem cells and the hematopoietic progenitor cells can easily enter the pores.

In the present invention, the “average length (Lb)” means one of “average length (Lb1)”, “average length (Lb2)”, and “average length (Lb3)” described below depending on the shapes formed by the outer boundaries of the bottoms of the pores.

In the present invention, when the shapes formed by the outer boundaries of the bottoms of the pores are polygons, the average length (Lb1) of the bottoms of the pores can be calculated by the same method for calculating the average length (La1) of the openings of the pores.

Specifically, after the length of each side of the polygon formed by the outer boundary of the bottom of one pore arbitrarily selected is measured, an average (Sb) of the lengths of all the sides is calculated. Such an average is calculated for ten pores arbitrarily selected, as an average (Sb₁), an average (Sb₂), an average (Sb₃), an average (Sb₄), an average (Sb₅), an average (Sb₆), an average (Sb₇), an average (Sb₈), an average (Sb₉), and an average (Sb₁₀). Next, an average of the averages (Sb₁) to (Sb₁₀) is calculated as the “average length (Lb1)”.

When the number of pores in the device for cell culture is a number (n) less than ten, an average of the averages (Sb₁) to (Sbₙ) is calculated as the “average length (Lb1)”.

The length of each side of the shape formed by the outer boundary of the bottom can be measured with a field-emission scanning electron microscope S-4700 (available from Hitachi High-Technologies Corporation).

In the present invention, when the shapes formed by the outer boundaries of the bottoms of the pores is not polygons, the average length (Lb2) of the bottoms of the pores can be calculated by the same method for calculating the average length (La2) of the openings of the pores.

Specifically, after the maximum length (Mb1) of the shape formed by the outer boundary of the bottom of one pore arbitrarily selected and the maximum length (Mb2) in the direction orthogonal to the maximum length (Mb1) are measured, an average (Ib) of the maximum length (Mb1) and the maximum length (Mb2) is calculated. Such an average is calculated for ten pores arbitrarily selected, as an average (Ib₁), an average (Ib₂), an average (Ib₃), an average (Ib₄), an average (Ib₅), an average (Ib₆), an average (Ib₇), an average (Ib₈), an average (Ib₉), and an average (Ib₁₀). Next, an average of the averages (Ib₁) to (Ib₁₀) is calculated as the “average length (Lb2)”.

When the number of pores in the device for cell culture is a number (n) less than ten, an average of the averages (Ib₁) to (Ibₙ) is calculated as the “average length (Lb2)”.

The maximum length (Mb1) of the shape formed by the outer boundary of the bottom and the maximum length (Mb2) in the direction orthogonal to the maximum length (Mb1) can be measured with a field-emission scanning electron microscope S-4700 (available from Hitachi High-Technologies Corporation).

The internal surface of the pore may be formed of the constituent material of the base material or surface treatment may be applied to the internal surface. It is preferable to apply surface treatment to the internal surface in terms of adhesiveness of hematopoietic stem cells or hematopoietic progenitor cells, or both the hematopoietic stem cells and the hematopoietic progenitor cells. Because hematopoietic stem cells or hematopoietic progenitor cells, or both the hematopoietic stem cells and the hematopoietic progenitor cells are typically non-adherent cells, surface treatment applied to the internal surface of the pore is advantageous because it facilitates hematopoietic stem cells or hematopoietic progenitor cells, or both the hematopoietic stem cells and the hematopoietic progenitor cells to adsorb to the internal surface and remain in the pore.

The material used for surface treatment of the internal surface of the pore is not particularly limited and may be appropriately selected depending on the intended purpose from materials commonly used for cell culture. Examples of the material include: hydrophilic polymers such as MPC polymers (2-methacryloyloxyethylphosphorylcholine), polyethylene glycol (PEG), and polyvinyl alcohol (PVA); natural polymers such as collagen, fibronectin, vitronectin, proteoglycan, gelatin, lectin, and polylysine; and inorganic materials such as hydroxyapatite. One of these materials may be used alone or two or more of these materials may be used in combination.

The method for applying surface treatment to the inside of the pore is not particularly limited and may be appropriately selected depending on the intended purpose from known surface treatment methods. Examples of the method include a dip coating method, a spray coating method, and a graft polymerization method.

It is preferable to apply the surface treatment to the internal surface of the pore in terms of adhesiveness of hematopoietic stem cells or hematopoietic progenitor cells, or both the hematopoietic stem cells and the hematopoietic progenitor cells. However, surface treatment may be applied all over the culture section or may be applied all over the device for cell culture.

### <<Other Members>>

The other members of the device for cell culture are not particularly limited and may be appropriately selected depending on the intended purpose. Examples of the other members include a handle, a joint, and a coupling member.

### Handle

The handle is a member used for taking out the device for cell culture from a culture container.

When the device for cell culture has the handle, there is an advantage that the device for cell culture has an excellent handleability because the device for cell culture can be easily taken out from the culture container.

The material of the handle is not particularly limited and may be appropriately selected depending on the intended purpose so long as the effect of the present invention is not spoiled. It is preferable that the material of the handle be the same as the material of the culture section, because it is easy to produce the device for cell culture. Hence, the plurality of pores may be provided in the surface of the handle as in the culture section. Moreover, the surface treatment may be applied to the surface of the handle as to the culture section.

The shape of the handle is not particularly limited and may be appropriately selected depending on the intended purpose so long as the device for cell culture can be taken out from the culture container. Examples of the shape of the handle include: pillar shapes such as round pillars, or polygonal prisms such as triangular prisms, quadrangular prisms, hexagonal prisms, and octagonal prisms that may have different lengths on the respective sides; pyramidal shapes such as circular cones, or truncated pyramids such as triangular pyramids, quadrangular pyramids, hexagonal pyramids, and octagonal pyramids that may have different lengths on the respective sides; sheet shapes or plate shapes of which surfaces have a circular shape such as a perfect circle (true circle) and an ellipse, or a polygonal shape such as a triangle, a quadrangle, a hexagon, and an octagon that may have different lengths on the respective sides; and combinations of these shapes.

The size of the handle is not particularly limited and may be appropriately selected depending on the intended purpose so long as the device for cell culture can be taken out from the culture container.

The position of the handle on the device for cell culture is not particularly limited and may be appropriately selected depending on the intended purpose so long as the device for cell culture can be taken out from the culture container. Examples of the position of the handle include all end positions on the sides or on the diameter of the culture section of the device for cell culture (positioning in a manner to surround the peripheral edge of the culture section), some end positions on the sides or on the diameter of the culture section of the device for cell culture, and any position that is surrounded by the sides or the diameter of the culture section of the device for cell culture (e.g., the center of the culture section).

It is preferable to provide the handle on the surface of the culture section including the pores. This is advantageous because the surface of the device for cell culture including the pores, i.e., the cell seeding surface can be easily identified and the device for cell culture has an excellent handleability when used as an insert.

The angle of the handle with respect to the cell seeding surface of the culture section is not particularly limited and may be appropriately selected depending on the intended purpose so long as the device for cell culture can be taken out from the culture container. When the device for cell culture is used as the insert, the angle may be appropriately designed depending on, for example, the shape of the culture container to which the device for cell culture is applied.

The number of handles on the device for cell culture is not particularly limited, may be appropriately selected depending on the intended purpose so long as the device for cell culture can be taken out from the culture container, and may be one or a plural number.

### Joint

The joint is a member that joins the culture section to the handle.

When the device for cell culture has the joint, there is an advantage that no step of joining the culture section to the handle is needed when producing the device for cell culture. When the joint is designed as a bendable shape or structure, there is an advantage that it is possible to easily produce the device for cell culture including the culture section and the handle simply by bending the joint.

The material of the joint is not particularly limited and may be appropriately selected depending on the intended purpose so long as the effect of the present invention is not spoiled. It is preferable that the material of the joint be the same as the material of the culture section because it is easy to produce the device for cell culture. Hence, the plurality of pores may be provided in the surface of the join as in the culture section. Moreover, the surface treatment may be applied to the surface of the joint as to the culture section.

The shape of the joint is not particularly limited and may be appropriately selected depending on the intended purpose so long as the joint can join the culture section to the handle. Examples of the shape of the joint include: pillar shapes such as round pillars, or polygonal prisms such as triangular prisms, quadrangular prisms, hexagonal prisms, and octagonal prisms that may have different lengths on the respective sides; pyramidal shapes such as circular cones, or truncated pyramids such as triangular pyramids, quadrangular pyramids, hexagonal pyramids, and octagonal pyramids that may have different lengths on the respective sides; sheet shapes or plate shapes of which surfaces have a circular shape such as a perfect circle (true circle) and an ellipse, or a polygonal shape such as a triangle, a quadrangle, a hexagon, and an octagon that may have different lengths on the respective sides; and combinations of these shapes.

The size of the joint is not particularly limited and may be appropriately selected depending on the intended purpose so long as the joint can join the culture section to the handle. However, it is preferable that the joint have a size that makes the area occupied by the joint in the culture section as small as possible. This is advantageous because a large area can be secured for culturing hematopoietic stem cells or hematopoietic progenitor cells, or both the hematopoietic stem cells and the hematopoietic progenitor cells in the culture section, and hematopoietic stem cells or hematopoietic progenitor cells, or both the hematopoietic stem cells and the hematopoietic progenitor cells can be efficiently proliferated.

The position of the joint on the device for cell culture is not particularly limited and may be appropriately selected depending on the intended purpose so long as the joint can join the culture section to the handle. Examples of the position of the joint include all end positions on the sides or on the diameter of the culture section of the device for cell culture (positioning in a manner to surround the peripheral edge of the culture section), some end positions on the sides or the diameter of the culture section of the device for cell culture, and any position that is surrounded by the sides or the diameter of the culture section of the device for cell culture (e.g., the center of the culture section).

The number of joints on the device for cell culture is not particularly limited, may be appropriately selected depending on the intended purpose so long as the culture section and the handle can be joined, and may be one or a plural number.

### Coupling Member

The coupling member is a member that is configured to couple one culture section to another culture section or one coupling member to another coupling member.

When the device for cell culture is used as the insert and applied to a culture container including a plurality of wells, and when the device for cell culture has no coupling member, devices for cell culture need to be inserted into the plurality of wells one by one and retrieved one by one after culturing, making handling bothersome. On the other hand, when the device for cell culture has the coupling member, there is an advantage that devices for cell culture can be inserted into the plurality of wells at a time and can be taken out from the plurality of wells at a time after culturing, providing an excellent handleability.

The material of the coupling member is not particularly limited and may be appropriately selected depending on the intended purpose so long as the effect of the present invention is not spoiled. It is preferable that the material of the coupling member be the same as the material of the culture section because it is easy to produce the device for cell culture.

The shape of the coupling member is not particularly limited and may be appropriately selected depending on the intended purpose so long as the coupling member can couple one culture section to another culture section or one coupling member to another coupling member. Examples of the shape of the coupling member include: pillar shapes such as round pillars, or polygonal prisms such as triangular prisms, quadrangular prisms, hexagonal prisms, and octagonal prisms that may have different lengths on the respective sides; pyramidal shapes such as circular cones, or truncated pyramids such as triangular pyramids, quadrangular pyramids, hexagonal pyramids, and octagonal pyramids that may have different lengths on the respective sides; sheet shapes or plate shapes of which surfaces have a circular shape such as a perfect circle (true circle) and an ellipse, or a polygonal shape such as a triangle, a quadrangle, a hexagon, and an octagon that may have different lengths on the respective sides; and combinations of these shapes.

The position, size, and number of the coupling member are not particularly limited and may be appropriately selected depending on the intended purpose so long as the coupling member can couple one culture section to another culture section or one coupling member to another coupling member. When the device for cell culture is used as the insert, the coupling member may be appropriately designed depending on, for example, the shape of the culture container to which the device for cell culture is applied.

The device for cell culture including the coupling member may have a shape that can cover wells of a culture container including a plurality of wells. Such a shape is advantageous because it is possible to recycle the culture container by taking out the device for cell culture from the culture container after culturing.

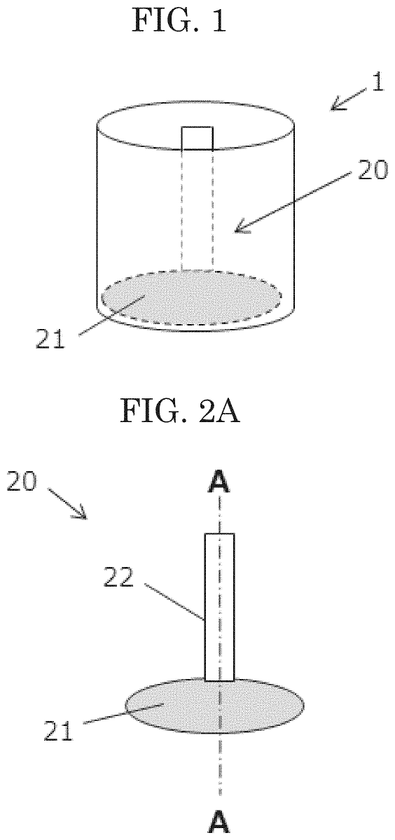

With reference to FIG. 1 to FIG. 4, an embodiment of the device for cell culture of the present invention when it is used as an insert for a 96-well plate will be described as one embodiment. However, the present invention is not limited to this embodiment.

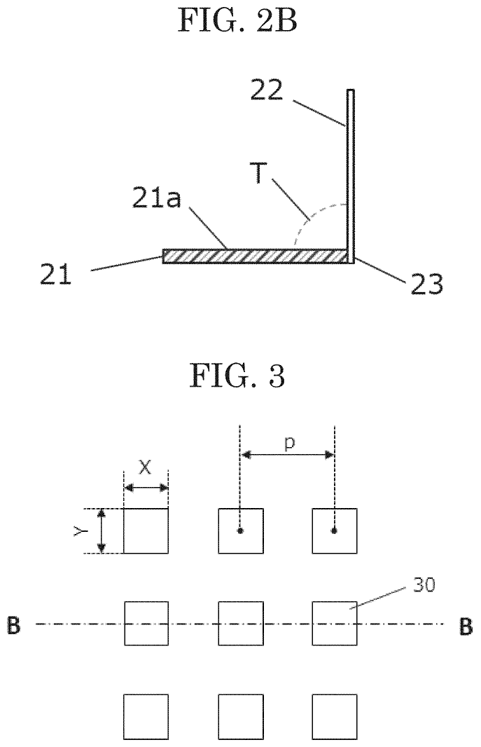

FIG. 1 is a view (perspective view) illustrating a state that a device 20 for cell culture including a culture section 21 having approximately the same circular shape as that of the bottom of a well 1 is inserted into one well 1 of a known 96-well plate and the culture section 21 is placed in contact with the bottom surface of the well 1. FIG. 2A is a view (perspective view) illustrating the whole appearance of the device 20 for cell culture placed in the well of FIG. 1. The device 20 for cell culture includes a handle 22. With the handle 22 picked with, for example, tweezers, the device 20 for cell culture is brought into or taken out from the well 1. FIG. 2B is a cross-sectional view of FIG. 2A taken along a line A-A. The culture section 21 and the handle 22 of the device 20 for cell culture are formed by bending at a joint 23 in a manner that a cell seeding surface 21a (i.e., a surface in which blind holes are formed as pores) of the culture section 21 comes to the opposite side from the surface of the culture section 21 contacting the bottom of the well 1 and an angle T formed between the cell seeding surface 21a and the handle 22 is less than 180 degrees, preferably about 90 degrees. With such a shape, the cell seeding surface 21a of the device 20 for cell culture can be set in the well 1 without fail.

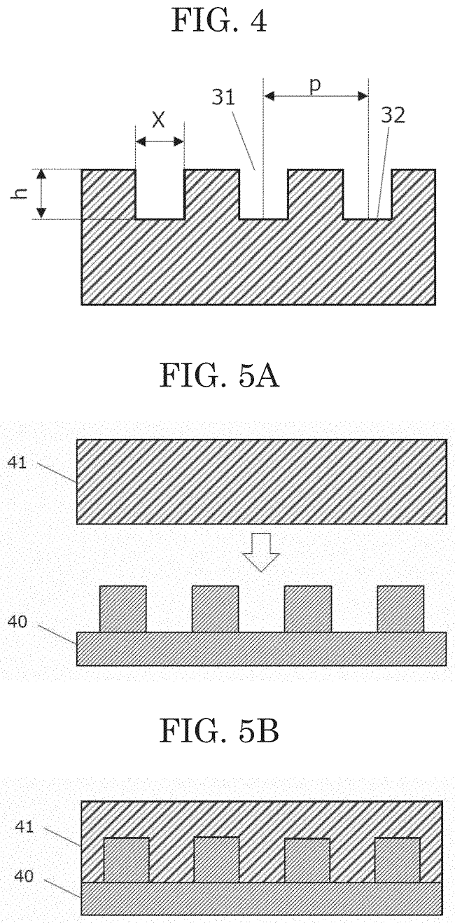

FIG. 3 is an enlarged view (top view) of a part of the cell seeding surface 21a of the culture section 21, illustrating an embodiment in which a plurality of pores 30 having rectangular openings are provided in one surface of the culture section 21. FIG. 4 is a cross-sectional view of FIG. 3 taken along a line B-B, where openings 31 are provided in the cell seeding surface 21a. FIG. 4 is a view illustrating an embodiment in which an opening 31 and a bottom 32 have approximately the same shape and size. In the present embodiment, the length X of one side of the opening 31 and the length Y of any other side in the same pore 30 may be the same as or different from each other. The preferable ranges of the average length (La), the average pitch P, the average depth H, and the aspect ratio [H/La] of the openings of a plurality of pores 30 are as described above. When hematopoietic stem cells or hematopoietic progenitor cells, or both the hematopoietic stem cells and the hematopoietic progenitor cells suspended in a culture liquid are seeded into the well 1 in a state that the device 20 for cell culture is placed in the well 1 as illustrated in FIG. 1, at least either the hematopoietic stem cells or the hematopoietic progenitor cells settle in the culture liquid, enter the inside of the pores 30 of the culture section 20, and adsorb to the inside of the pores 30. Here, because at least either the hematopoietic stem cells or the hematopoietic progenitor cells can enter the inside of the pores 30 at an appropriate density, the cells are mutually influenced and amplified with their undifferentiated state maintained.

### <Producing Method>

The method for producing the device for cell culture is not particularly limited and may be appropriately selected depending on the intended purpose. Examples of the method include a method of producing a plurality of pores in a surface of a base material of which Young's modulus measured according to JIS K 7161-1 and JIS K 7161-2 is at least 3 GPa, and further producing a handle and a joint as needed.

The method for producing a plurality of pores in a surface of the base material is not particularly limited and may be appropriately selected from known molding methods depending on, for example, the constituent material of the base material used. Examples of the method include a heat compression molding method, a transfer molding method, an injection molding method, an extrusion molding method (e.g., a T die method), a laminate molding method, and a vacuum molding method.

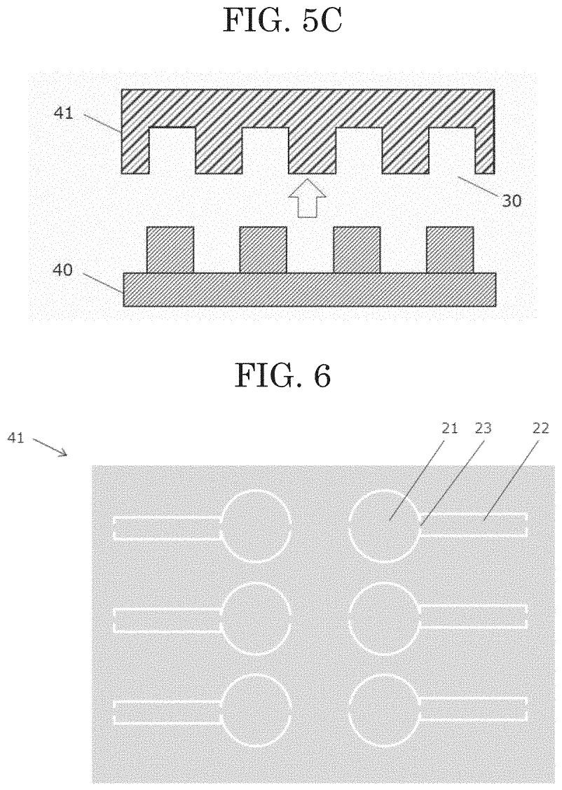

An example of the method for producing the device for cell culture of the present invention will be specifically described below with reference to FIG. 5A to FIG. 6. However, the method for producing the device for cell culture is not limited to this method.

First, a master plate 40 having a surface structure that is inverted from the shapes of the plurality of pores in the surface of the culture section is produced.

The material of the master plate 40 is not particularly limited and may be appropriately selected depending on the intended purpose. Examples of the material of the master plate 40 include metals, glass, silicon, and resins. One of these materials may be used alone or two or more of these materials may be used in combination.

Examples of the metals include iron-based metals, aluminum-based metals, copper-based metals, and stainless steels such as SUS. One of these metals may be used alone or two or more of these metals may be used in combination. Among these metals, iron-based metals or stainless steels plated with electroless Ni—P coating and oxygen-free copper are preferable.

As the resin, a resin that has a higher glass transition temperature (Tg) than the constituent material of the base material is preferable.

The method for processing the master plate 40 to have the surface structure is not particularly limited and may be appropriately selected from known methods.

Examples of the method include machine cutting, laser lithography, laser interference lithography, electron beam lithography, and etching. Among these methods, machine cutting is preferable, and precision machine processing using a single crystal diamond is more preferable.

When a film-shaped thermoplastic resin (hereinafter, may be referred to as “resin film”) is used as the constituent material of the base material, the resin film 41 is aligned with the master plate 40 having the surface structure (convexed shapes) inverted from the concaves serving as the plurality of pores in the surface of the culture section, and pressed against the master plate 40 while being heated as illustrated in FIG. 5A. As a result, the convexed surface structure of the master plate 40 is transferred to the resin film 41 as a pressure-bonded pattern. The heating temperature for transferring is not particularly limited, may be appropriately selected depending on the intended purpose, and is preferably a temperature higher than the glass transition temperature (Tg) of the resin film 41.

Next, as illustrated in FIG. 5B, the resin film 41 is sufficiently pressed against the master plate 40. The pressure for transferring is not particularly limited and may be appropriately selected depending on the intended purpose.

Next, as illustrated in FIG. 5C, the plate is cooled when the convexed surface structure of the master plate 40 is transferred to the resin film 41, to release the resin film 41 from the master plate 40. As a result, a plurality of pores 30 can be formed in the surface of the resin film 41, and the resin film having the plurality of pores 30 can be used as the culture section.

Although not illustrated in the drawings, a case where the ultraviolet-curable resin is used as the constituent material of the base material instead of the resin film 41 will also be described.

First, the ultraviolet-curable resin is applied to a master plate having a surface structure inverted from the shapes of the plurality of pores in the surface of the culture section. Next, a transparent resin film (e.g., a PET film; COSMOSHINE A4300 available from Toyobo Co., Ltd.) is pasted over a surface of the ultraviolet-curable resin opposite to the surface contacting the master plate, and the ultraviolet-curable resin is cured by irradiation with ultraviolet rays through the transparent resin film. Next, when the ultraviolet-curable resin cured is released from the master plate, a plurality of pores can be formed in the surface of the ultraviolet-curable resin, and the ultraviolet-curable resin having the plurality of pores can be used as the culture section.

FIG. 6 is a top view of the resin film 41 serving as the base material including culture sections including the plurality of pores that are produced in the manner described above (i.e., a view illustrating the surface including the plurality of pores). When a culture section 21 is punched out from the resin film 41, a culture section 21 including the plurality of pores having a desired shape can be obtained. Here, as needed, a handle 22 having a desired shape may also be punched out. Here, the culture section 21 and the handle 22 may be punched out separately or may be punched out simultaneously. It is advantageous to simultaneously punch out the culture section 21 and the handle 22 in a manner to have a joint 23, because it is possible to produce the device for cell culture having the culture section 21 and the handle 22 easily by bending the joint 23.

FIG. 6 illustrates the device for cell culture having an approximately rectangular handle 22 at one of the end positions on the diameter of a circular culture section 21. However, for example, the size, position, and shape of the culture section 21 and the handle 22 are not limited to this example.

FIG. 6 illustrates a method for producing the device for cell culture having the culture section 21, the handle 22, and the joint 23 all at a time from one resin film 41 including a plurality of pores. Therefore, not only the culture section 21, but also the handle 22 and the joint 23 may have the same surface structure as that of the culture section 21 (i.e., the plurality of pores). However, the handle and the joint of the device for cell culture of the present invention may be produced from a base material different from that of the culture section.

### <Applications>

Because the device for cell culture can be produced easily at low costs and can efficiently proliferate hematopoietic stem cells or hematopoietic progenitor cells, or both the hematopoietic stem cells and the hematopoietic progenitor cells in vitro while maintaining the self-replication ability and the multipotency thereof, the device for cell culture can be suitably used as a scaffold for culturing and proliferating hematopoietic stem cells or hematopoietic progenitor cells, or both the hematopoietic stem cells and the hematopoietic progenitor cells in vitro. The device for cell culture can also be suitably used in a cell culturing method of the present invention described below. Moreover, the device for cell culture can be suitably used for, for example, studies about, for example, maintenance, proliferation, and differentiation of hematopoietic stem cells.

After the device for cell culture is used for culturing hematopoietic stem cells or hematopoietic progenitor cells, or both the hematopoietic stem cells and the hematopoietic progenitor cells, as needed, the device for cell culture may be used continuously for culturing hematopoietic cells into which hematopoietic stem cells or hematopoietic progenitor cells, or both the hematopoietic stem cells and the hematopoietic progenitor cells have differentiated with addition of, for example, a factor that differentiates hematopoietic stem cells or hematopoietic progenitor cells, or both the hematopoietic stem cells and the hematopoietic progenitor cells into desired hematopoietic cells.

The factor that differentiates hematopoietic stems cells or hematopoietic progenitor cells into hematopoietic cells is not particularly limited and may be appropriately selected from known factors. Examples of the factor include a granulocyte-monocyte colony-stimulating factor (GM-CSF), a granulocyte colony stimulating factor (G-CSF), a macrophage colony stimulating factor (M-CSF), thrombopoietin (TPO), erythropoietin (EPO), oncostatin M, and various interleukins (IL).

The culture container is not particularly limited and may be appropriately selected depending on the intended purpose from known culture containers. Examples of the culture container include: for example, 384-well, 192-well, 96-well, 48-well, 24-well, 12-well, and 6-well multi-well microplates; for example, 8-well, 4-well, and 2-well multi-well chambers or square dishes; round cell culture dishes having a diameter of, for example, 35 mm, 60 mm, 100 mm, and 150 mm; and flask-type culture containers.

The material of the culture container is not particularly limited and may be appropriately selected depending on the intended purpose from materials of known culture containers. Examples of the material of the culture container include glass, polystyrene, polypropylene, and polycarbonate.

### <<Hematopoietic Stem Cells and Hematopoietic Progenitor Cells>>

Hematopoietic stem cells are cells that have both multipotency to differentiate into hematopoietic cells of all lineages such as white blood cells (e.g., neutrophils, eosinophils, basophils, lymphocytes, monocytes, and macrophages), red blood cells, platelets, mast cells, and dendritic cells, and self-replication ability to replicate themselves while maintaining the multipotency, and are also referred to as “long-term hematopoietic stem cells”.

Hematopoietic progenitor cells are cells that have multipotency to differentiate into hematopoietic cells of various lineages although having no self-replication ability like hematopoietic stem cells.

The source of hematopoietic stem cells or hematopoietic progenitor cells, or both the hematopoietic stem cells and the hematopoietic progenitor cells to be cultured with the device for cell culture is not particularly limited and may be appropriately selected depending on, for example, the purpose of use after culture. Examples of the source include: primates such as human, monkey, and marmoset; rodents such as mouse, rat, and hamster; birds such as rooster or hen; lagomorphas such as rabbit; ungulates such as pig, sheep, bovine, goat, and horse; and order carnivora such as dog and cat.

Among these sources, at least either human-derived hematopoietic stem cells or human-derived hematopoietic progenitor cells are preferable in terms of application to treatment of blood cancers such as leukemia, malignant lymphoma, and multiple myeloma.

The tissue as the source of hematopoietic stem cells or hematopoietic progenitor cells, or both the hematopoietic stem cells and the hematopoietic progenitor cells to be cultured with the device for cell culture is not particularly limited and may be appropriately selected depending on the intended purpose. Examples of the tissue include bone marrow, umbilical cord blood, peripheral blood, and liver. One kind of hematopoietic stem cells or hematopoietic progenitor cells, or both the hematopoietic stem cells and the hematopoietic progenitor cells derived from these tissues may be used alone or two or more kinds of hematopoietic stem cells or hematopoietic progenitor cells, or both the hematopoietic stem cells and the hematopoietic progenitor cells derived from these tissues may be used in combination.

Hematopoietic stem cells or hematopoietic progenitor cells, or both the hematopoietic stem cells and the hematopoietic progenitor cells to be cultured with the device for cell culture may be primary culture cells isolated from the tissue or may be serially subcultured cells, or may be the primary culture cells or the serially subcultured cells that are cryopreserved and then melted. One of these kinds of cells may be used alone or two or more of these kinds of cells may be used in combination.

The method for isolating hematopoietic stem cells or hematopoietic progenitor cells, or both the hematopoietic stem cells and the hematopoietic progenitor cells from the tissue is not particularly limited and may be appropriately selected depending on the intended purpose. Examples of the method include a method of isolating hematopoietic stem cells or hematopoietic progenitor cells, or both the hematopoietic stem cells and the hematopoietic progenitor cells from the tissue, observing expression of a cell surface marker specific to hematopoietic stem cells or hematopoietic progenitor cells, or both the hematopoietic stem cells and the hematopoietic progenitor cells (a hematopoietic stem cell-specific surface marker or a hematopoietic progenitor cell-specific surface marker) as an indicator.

The method for isolating hematopoietic stem cells or hematopoietic progenitor cells, or both the hematopoietic stem cells and the hematopoietic progenitor cells from the tissue, observing expression of a cell surface marker specific to hematopoietic stem cells or hematopoietic progenitor cells, or both the hematopoietic stem cells and the hematopoietic progenitor cells is not particularly limited and may be appropriately selected from known methods. Examples of the method include a method of using an antibody to the hematopoietic stem cell-specific surface marker or to the hematopoietic progenitor cell-specific surface marker and isolating cells having the characteristic of the hematopoietic stem cell-specific surface marker or the hematopoietic progenitor cell-specific surface marker with, for example, a cell sorter and magnetic beads.

The cell surface marker specific to hematopoietic stem cells or hematopoietic progenitor cells, or both the hematopoietic stem cells and the hematopoietic progenitor cells may be appropriately selected depending on the animal species from which hematopoietic stem cells or hematopoietic progenitor cells, or both the hematopoietic stem cells and the hematopoietic progenitor cells are derived.

Human-derived hematopoietic stem cells are typically CD34-positive (+), CD90 (Thy1)-positive (+), and CD45RA-negative (−). In addition to these cell surface markers, cell surface markers such as CD38-negative (−) and CD49 (CD490-positive (+) may also be used in combination.

Human-derived hematopoietic progenitor cells are typically CD34-positive (+), CD38-negative (−), CD45RA-negative (−), and CD90 (Thy1)-negative (−).

Examples of mouse-derived hematopoietic stem cells include long-term hematopoietic stem cells (LT-HSC: long-term HSC), intermediate-term hematopoietic stem cells (IT-HSC: intermediate-term HSC), and short-term hematopoietic stem cells (ST-HSC: short-term HSC).

Examples of the markers of the long-term hematopoietic stem cells (LT-HSC) include Sca-1-positive (+), CD117-positive (+), CD34-negative (−), CD48-negative (−), CD49b^{low}, CD135-negative (−), and CD150-positive (+).

Examples of the markers of the intermediate-term hematopoietic stem cells (IT-HSC) include Sca-1-positive (+), CD117-positive (+), CD34-negative (−), CD49^{high}, CD135-negative (−), and CD150-positive (+).

Examples of the markers of the short-term hematopoietic stem cells (ST-HSC) include Sca-1-positive (+), CD117-positive (+), CD34-positive (+), CD48-negative (−), CD135-negative (−), and CD150-negative (−).

Mouse-derived hematopoietic progenitor cells are Sca-1-positive (+), CD117-positive (+), CD34-positive (+), CD48-negative (−), and CD135-positive (+).

### (Cell Culturing Method)

A cell culturing method of the present invention is a method of culturing hematopoietic stem cells or hematopoietic progenitor cells, or both the hematopoietic stem cells and the hematopoietic progenitor cells using the device for cell culture of the present invention described above.

The cell culturing method preferably includes a seeding step and a culturing step, may include a collecting step, a retrieving step, and a serial subculture step, and may further include other step as needed.

### <Seeding Step>

The seeding step is a step of seeding a least either hematopoietic stem cells or hematopoietic progenitor cells into the device for cell culture.

The seeding step is performed in order to introduce hematopoietic stem cells or hematopoietic progenitor cells, or both the hematopoietic stem cells and the hematopoietic progenitor cells to the inside of the plurality of pores of the device for cell culture.

In the seeding step, hematopoietic stem cells or hematopoietic progenitor cells, or both the hematopoietic stem cells and the hematopoietic progenitor cells need not be introduced into the inside of all of the plurality of pores but need only to be introduced into the inside of at least one pore. However, in terms of cell proliferation efficiency, the number of pores into which hematopoietic stem cells or hematopoietic progenitor cells, or both the hematopoietic stem cells and the hematopoietic progenitor cells are introduced is preferably as large as possible.

The number of hematopoietic stem cells or hematopoietic progenitor cells, or both the hematopoietic stem cells and the hematopoietic progenitor cells to be introduced into one pore in the seeding step is not particularly limited and may be appropriately selected depending on, for example, the average length (La), the average depth (H), the aspect ratio (H/La), and the average pitch (P) of the openings of the pores.

The number of hematopoietic stem cells or hematopoietic progenitor cells, or both the hematopoietic stem cells and the hematopoietic progenitor cells to be introduced into one pore can be adjusted by the number of hematopoietic stem cells or hematopoietic progenitor cells, or both the hematopoietic stem cells and the hematopoietic progenitor cells to be seeded first in the seeding step.

As hematopoietic stem cells or hematopoietic progenitor cells, or both the hematopoietic stem cells and the hematopoietic progenitor cells used in the seeding step, the same cells as described in the section “<<Hematopoietic stem cells and hematopoietic progenitor cells>>” described above can be used.

The method for seeding hematopoietic stem cells or hematopoietic progenitor cells, or both the hematopoietic stem cells and the hematopoietic progenitor cells into the device for cell culture is not particularly limited and may be appropriately selected depending on the intended purpose from known cell seeding methods. It is preferable to bring hematopoietic stem cells or hematopoietic progenitor cells, or both the hematopoietic stem cells and the hematopoietic progenitor cells into a single-cell state before seeding. Specific examples of the seeding method include a method of suspending hematopoietic stem cells or hematopoietic progenitor cells, or both the hematopoietic stem cells and the hematopoietic progenitor cells brought into a single-cell state in a solution such as a culture liquid and dropping the solution into the device for cell culture using, for example, a pipette.

The method for bringing hematopoietic stem cells or hematopoietic progenitor cells, or both the hematopoietic stem cells and the hematopoietic progenitor cells into a single-cell state is not particularly limited and may be appropriately selected from known methods. Examples of the method include a method of applying physical treatment such as pipetting.

The number of hematopoietic stem cells or hematopoietic progenitor cells, or both the hematopoietic stem cells and the hematopoietic progenitor cells to be seeded into the device for cell culture is not particularly limited and may be appropriately selected depending on, for example, the size of the culture section of the device for cell culture, the size and number of the pores in the culture section, and the intended number of hematopoietic stem cells or hematopoietic progenitor cells, or both the hematopoietic stem cells and the hematopoietic progenitor cells after cultured.

For example, when the device for cell culture is used as an insert for a known 96-well plate and when the shape and size of the culture section of the device for cell culture are approximately the same as the shape and size of the bottom surface of a well of the 96-well plate, it is preferable to seed hematopoietic stem cells or hematopoietic progenitor cells, or both the hematopoietic stem cells and the hematopoietic progenitor cells by from 1,000 cells/well through 10,000 cells/well, and more preferably by from 1,000 cells/well through 5,000 cells/well.

### <Culturing Step>

The culturing step is a step of culturing hematopoietic stem cells or hematopoietic progenitor cells, or both the hematopoietic stem cells and the hematopoietic progenitor cells seeded in the seeding step.

The culturing step is performed in order to efficiently proliferate hematopoietic stem cells or hematopoietic progenitor cells, or both the hematopoietic stem cells and the hematopoietic progenitor cells in vitro while maintaining the self-replication ability and the multipotency thereof.

The culture medium (culture liquid) used for culturing hematopoietic stem cells or hematopoietic progenitor cells, or both the hematopoietic stem cells and the hematopoietic progenitor cells is not particularly limited and may be appropriately selected from known culture media.

Typically, hematopoietic stem cells or hematopoietic progenitor cells, or both the hematopoietic stem cells and the hematopoietic progenitor cells are cultured in a culture medium containing, for example, cytokines such as a stem cell actor (SCF), thrombopoietin (TPO), Flt-3 ligand (FL), interleukins (IL)-3, IL-6, and IL-11, and serum albumin such as bovine serum albumin (BSA) in order to maintain the undifferentiation property of hematopoietic stem cells or hematopoietic progenitor cells, or both the hematopoietic stem cells and the hematopoietic progenitor cells. However, the culture medium is not limited to this culture medium.

The conditions (e.g., temperature, carbon dioxide (CO₂) concentration, and oxygen concentration) for culturing hematopoietic stem cells or hematopoietic progenitor cells, or both the hematopoietic stem cells and the hematopoietic progenitor cells are not particularly limited and may be appropriately selected from known conditions.

The temperature for culturing hematopoietic stem cells or hematopoietic progenitor cells, or both the hematopoietic stem cells and the hematopoietic progenitor cells is typically from 30 degrees C. through 40 degrees C. and preferably about 37 degrees C.

The CO₂ concentration for culturing hematopoietic stem cells or hematopoietic progenitor cells, or both the hematopoietic stem cells and the hematopoietic progenitor cells is typically from 1% by volume through 10% by volume and preferably from 2% by volume through 5% by volume.

It is possible to adjust the conditions for culturing hematopoietic stem cells or hematopoietic progenitor cells, or both the hematopoietic stem cells and the hematopoietic progenitor cells with a commercially available cell culturing device such as an incubator.

The culture time for culturing hematopoietic stem cells or hematopoietic progenitor cells, or both the hematopoietic stem cells and the hematopoietic progenitor cells is not particularly limited and may be appropriately selected depending on, for example, the intended number of hematopoietic stem cells or hematopoietic progenitor cells, or both the hematopoietic stem cells and the hematopoietic progenitor cells after cultured.

When the culture time in the culturing step is a long period, the culture medium may be appropriately replaced with new one or circulated, or may be subjected to the serial subculture step described below.

It is possible to confirm that the cells cultured in the culturing step are hematopoietic stem cells or hematopoietic progenitor cells, or both the hematopoietic stem cells and the hematopoietic progenitor cells by confirming expression of a cell surface marker specific to hematopoietic stem cells or hematopoietic progenitor cells, or both the hematopoietic stem cells and the hematopoietic progenitor cells described in the section <<Hematopoietic stem cells and hematopoietic progenitor cells>> described above.

As the method for confirming expression of the cell surface marker, an immunostaining method, a method for measuring enzyme activity, and a real-time RT-PCR method may be used in addition to the above-described method using, for example, a cell sorter and magnetic beads.

### <Collecting Step>

The collecting step is a step of collecting hematopoietic stem cells or hematopoietic progenitor cells, or both the hematopoietic stem cells and the hematopoietic progenitor cells cultured in the culturing step.

When the cell culturing method includes the retrieving step, the collecting step can be performed on the device for cell culture retrieved in the retrieving step.

The method for collecting hematopoietic stem cells or hematopoietic progenitor cells, or both the hematopoietic stem cells and the hematopoietic progenitor cells cultured in the culturing step is not particularly limited and may be appropriately selected from known cell collecting methods. Examples of the method include a pipetting method, and a method of applying vibration by shaking or clapping the device for cell culture (or a culture container when the device for cell culture is used as the insert).

The cells floated or stripped from the device for cell culture by the method described above can be collected together with the culture liquid.

### <Retrieving Step>

The retrieving step is a step of retrieving the device for cell culture together with at least either cultured hematopoietic stem cells or cultured hematopoietic progenitor cells from a culture container after the culturing step or retrieving the device for cell culture after the collecting step from a culture container after the culturing step, when the device for cell culture is used as the insert.

The method for retrieving the device for cell culture is not particularly limited and may be appropriately selected depending on the intended purpose. Examples of the method include a retrieving method using tweezers.

In the retrieving step, it is suitable to use the handle or the coupling member of the device for cell culture.

### <Serial Subculture Step>

The serial subculture step is a step of serially culturing hematopoietic stem cells or hematopoietic progenitor cells, or both the hematopoietic stem cells and the hematopoietic progenitor cells cultured in the culturing step.

The method for serial subculture is not particularly limited and may be appropriately selected depending on the intended purpose. It is preferable to perform the serial subculture step by repeating the collecting step, the seeding step, the culturing step, and as needed, the retrieving step. The serial subculture step may be performed only once or a plurality of times.

When the cell culturing method is performed only once, the number of cells to be obtained is limited. When the culture time is a long period, there are risks of cell aging and cell death due to, for example, cell density growth, and the self-replication ability and the multipotency of hematopoietic stem cells or hematopoietic progenitor cells, or both the hematopoietic stem cells and the hematopoietic progenitor cells may not be maintained. Therefore, the cell culturing method including the serial subculture step is advantageous because it is possible to proliferate hematopoietic stem cells or hematopoietic progenitor cells, or both the hematopoietic stem cells and the hematopoietic progenitor cells even more while maintaining the self-replication ability and the multipotency of hematopoietic stem cells or hematopoietic progenitor cells, or both the hematopoietic stem cells and the hematopoietic progenitor cells.

### <Applications>

Because the cell culturing method can efficiently proliferate hematopoietic stem cells or hematopoietic progenitor cells, or both the hematopoietic stem cells and the hematopoietic progenitor cells in vitro while maintaining the self replication ability and the multipotency thereof, the cell culturing method can be suitably used for in vitro culture of hematopoietic stem cells or hematopoietic progenitor cells, or both the hematopoietic stem cells and the hematopoietic progenitor cells.

Hematopoietic stem cells or hematopoietic progenitor cells, or both the hematopoietic stem cells and the hematopoietic progenitor cells obtained by the cell culturing method are suitably used for, for example, treatment of blood cancers such as leukemia, malignant lymphoma, and multiple myeloma and studies about maintenance, proliferation, and differentiation of hematopoietic stem cells. Hematopoietic stem cells or hematopoietic progenitor cells, or both the hematopoietic stem cells and the hematopoietic progenitor cells obtained by the cell culturing method may be cryopreserved by a known method.

### EXAMPLES

The present invention will be described below by way of Examples, Comparative Examples, and Test Examples. The present invention should not be construed as being limited to these Examples and Test Examples.

### Example 1

### <Production of Mold>

Martensite-based stainless steel (SUS420J2, with a width of 70 mm, a length of 70 mm, and a thickness of 8 mm) was used as a mold material. A surface of the mold material to be processed was plated with electroless Ni—P, to form a plated layer having a thickness of 150 micrometers. Next, using an ultraprecision machine tool, the plated surface was cut and smoothed with a single crystal diamond bite having a curvature radius of 5 mm. Next, a range within the plated surface having a width of 20 mm and a length of 20 mm was processed to have a minutely convexed structure (in a grid pattern) with an approximately rectangular single crystal diamond bite having a tip width of 40.3 micrometers and a tip angle of 8 degrees (at one side). As the minutely convexed structure, specifically, a group of rectangular parallelepiped pillars having a width of 47 micrometers, a length of 47 micrometers, a height of 50 micrometers, a width-direction pitch (i.e., a center-to-center distance between convexes in the width direction of the convexed structure) of 87.3 micrometers, and a pitch (i.e., a center-to-center distance between convexes in the length direction of the convexed structure) of 87.3 micrometers were formed.

The minutely convexed structure is a structure inverted from the structure to be formed in a surface of the culture section of the device for cell culture to be obtained finally.

### <Molding of Base Material of Device for Cell Culture>

Abase material used for the device for cell culture was molded using the mold described above by a heat compression molding method.

Specifically, a non-stretched polystyrene film (hereinafter, may be referred to simply as “polystyrene film”) having a thickness of 0.1 mm was used as the base material used for the device for cell culture. The polystyrene film was placed on the minutely convexed structured-surface of the mold, and a SUS plate (SUS304, having a width of 70 mm, a length of 70 mm, and a thickness of 1 mm) was placed on the polystyrene film. The polystyrene film and the SUS plate were both heated to 130 degrees C. in a non-pressurized state. When the temperature reached 130 degrees C., a pressure of 0.3 MPa was applied for 300 s. Subsequently, in order to solidify the polystyrene film, the temperature was lowered to 40 degrees C. with the pressurization maintained. Next, the resultant was depressurized to a non-loaded state, and then the polystyrene film was released from the mold. In this way, the base material used for the device for cell culture was obtained.

### <Punching>

A circular culture section having a diameter of 6 mm and a rectangular handle having a length of 8 mm and a width of 2 mm were punched out from the obtained base material used for the device for cell culture, using a carbon dioxide laser (see FIG. 6).

### Example 2

A device for cell culture of Example 2 was obtained in the same manner as in Example 1, except that unlike in <Production of mold> in Example 1, the minutely convexed structure of the mold was changed to a group of rectangular parallelepiped pillars having a width of 15 micrometers, a length of 15 micrometers, a height of 15 micrometers, a pitch (a center-to-center distance between convexes in the width direction of the convexed structure) of 30.0 micrometers, and a pitch (a center-to-center distance between convexes in the length direction of the convexed structure) of 30.0 micrometers.

### Example 3

A device for cell culture of Example 3 was obtained in the same manner as in Example 1, except that unlike in <Production of mold> in Example 1, the minutely convexed structure of the mold was changed to a group of rectangular parallelepiped pillars having a width of 100 micrometers, a length of 100 micrometers, a height of 90 micrometers, a pitch (a center-to-center distance between convexes in the width direction of the convexed structure) of 200.0 micrometers, and a pitch (a center-to-center distance between convexes in the length direction of the convexed structure) of 200.0 micrometers, and the base material used for the device for cell culture was changed to a non-stretched polystyrene film having a thickness of 0.2 mm.

### Example 4

A device for cell culture of Example 4 was obtained in the same manner as in Example 1, except that unlike in <Production of mold> in Example 1, the minutely convexed structure of the mold was changed to a group of rectangular parallelepiped pillars having a width of 30 micrometers, a length of 30 micrometers, a height of 30 micrometers, a pitch (a center-to-center distance between convexes in the width direction of the convexed structure) of 64.1 micrometers, and a pitch (a center-to-center distance between convexes in the length direction of the convexed structure) of 64.1 micrometers.

### Example 5

A device for cell culture of Example 5 was obtained in the same manner as in Example 1, except that unlike in <Production of mold> in Example 1, the minutely convexed structure of the mold was changed to a group of rectangular parallelepiped pillars having a width of 80 micrometers, a length of 80 micrometers, a height of 80 micrometers, a pitch (a center-to-center distance between convexes in the width direction of the convexed structure) of 160 micrometers, and a pitch (a center-to-center distance between convexes in the length direction of the convexed structure) of 160 micrometers.

### Example 6

A device for cell culture of Example 6 was obtained in the same manner as in Example 1, except that unlike in Example 1, the base material used for the device for cell culture was changed to polycarbonate having a thickness of 0.1 mm.

### Comparative Example 1

A device for cell culture of Comparative Example 1 was obtained in the same manner as in Example 1, except that unlike in Example 1, the base material used for the device for cell culture was changed to low-density polyethylene having a thickness of 0.1 mm.

Data of the devices for cell culture of Examples 1 to 6 and Comparative Example 1 are presented collectively in Table 1 below.

TABLE 1

| | | | | | | | Comp. |
|---|---|---|---|---|---|---|---|
| | Ex. 1 | Ex. 2 | Ex. 3 | Ex. 4 | Ex. 5 | Ex. 6 | Ex. 1 |
| Constituent | Poly- | Poly- | Poly- | Poly- | Poly- | Poly- | Low-density |
| material | styrene | styrene | styrene | styrene | styrene | carbonate | poly- |
| of base | | | | | | | ethylene |
| material | | | | | | | |
| Young's | 3.00 | 3.00 | 3.00 | 3.00 | 3.00 | 5.10 | 0.35 |
| modulus (GPa) | | | | | | | |
| Thickness (mm) | 0.1 | 0.1 | 0.2 | 0.1 | 0.1 | 0.1 | 0.1 |
| Average length | 47 | 15 | 100 | 30 | 80 | 47 | 47 |
| [La] of openings | | | | | | | |
| (micrometer) | | | | | | | |
| Average depth | 50 | 15 | 90 | 30 | 80 | 50 | 50 |
| [H] | | | | | | | |
| (micrometer) | | | | | | | |
| Average pitch | 87.3 | 30.0 | 200.0 | 64.1 | 160.0 | 87.3 | 87.3 |
| [P] | | | | | | | |
| (micrometer) | | | | | | | |
| Aspect ratio | 1.1 | 1.0 | 0.9 | 1.0 | 1.0 | 1.1 | 1.1 |
| [H/La] | | | | | | | |

The Young's modulus, the average length [La1] of openings of pores, the average depth [H], and the average pitch [P] presented in Table 1 are values measured in the manners described below.

### Young's Modulus

The Young's modulus of the base materials (polystyrene) used in Examples 1 to 5, the base material (polycarbonate) used in Example 6, and the base material (low-density polyethylene) used in Comparative Example 1 was measured according to JIS K 7161-1 and JIS K 7161-2.

### Average Length [La1] of Openings

Arbitrary ten pores in the surface of the culture section were selected, the lengths of the sides of the shapes formed by the outer boundaries of the openings of the ten pores were measured with a field-emission scanning electron microscope S-4700 (obtained from Hitachi High-Technologies Corporation), and averages (Sa₁) to (Sa₁₀) of the lengths were calculated. Next, an average of the averages (Sa₁) to (Sa₁₀) was calculated as the average length [La] of the openings of the pores.

### Average Depth [H]

Arbitrary ten pores in the surface of the culture section were selected, the depth of the pores from a surface of the base material serving as a reference surface to the bottoms of the pores in the thickness direction of the base material of the culture section (or the length of the cross-sections of the pores in the thickness direction) (h) was measured with a field-emission scanning electron microscope S-4700 (obtained from Hitachi High-Technologies Corporation), and an average of the depths (h) of the ten pores was calculated as the average depth [H] of the pores.

### Average Pitch [P]

The minimum center-to-center distance (pitch (p)) between the center of the shape formed by the outer boundary of the opening of one pore arbitrarily selected in the surface of the culture section and the center of the shape formed by the outer boundary of the opening of another pore adjoining the one pore arbitrarily selected was measured with a field-emission scanning electron microscope S-4700 (obtained from Hitachi High-Technologies Corporation). This minimum center-to-center distance was measured for ten pores arbitrarily selected, and an average of the minimum center-to-center distances of the ten pores was calculated as the average pitch [P].

### Preparation Example 1: Preparation of CD34-Positive Cells

CD34-positive cells used in Test Examples described below were prepared in the manner described below.

FICOLL (obtained from Nacalai Tesque, Inc.) (12.5 mL) was dispensed into tubes (with a volume of 50 mL). Human-derived umbilical cord blood (250 mL) was poured into a sterilized culture bottle (with a volume of 500 mL), and then PBS (250 mL) was added and mixed with the umbilical cord blood, to obtain a diluted liquid of the umbilical cord blood. Next, the diluted liquid of the umbilical cord blood (37.5 mL) was stacked over the FICOLL in each tube. The tubes were centrifuged at 1,500 rpm at 25 degrees C. for 25 minutes. The top layers separated as a result (i.e., layers containing blood plasma) were removed, and then layers (liquid phases) containing mononucleosis and CD34-positive cells were collected and transferred to new tubes (with a volume of 50 mL). An Iscove's Modified Dulbecco's Medium (IMDM, obtained from GE Healthcare Inc.) (25 mL) was added to the obtained liquids (25 mL) containing mononucleosis and CD34-positive cells, the resultants were centrifuged at 1,500 rpm at 4 degrees C. for 10 minutes, and the supernatants were removed. A staining buffer (SM; PBS containing 2% by volume fetal bovine serum (FBS, obtained from Sigma-Aldrich Co. LLC)) (2 mL) was added to the obtained precipitates to re-suspend the precipitates, and the contents in all of the tubes were combined in one tube.

Then, the number of cells obtained was measured with a Burker-Turk counting chamber. As a result, the number of cells was 1.10×10⁹ cells.

The cells suspended in the SM were centrifuged at 1,500 rpm at 4 degrees C. for 10 minutes, and the supernatant was removed. A staining medium (SM) was added to the obtained precipitate, the number of cells was adjusted to 1×10⁸ cells/50 microliters, and a FcR blocking reagent (obtained from IMMUNOSTEP Inc.) (50 microliters) and CD34 microbeads (CD34 MICROBEAD KIT ULTRAPURE, human, obtained from Miltenyi Biotec GmbH) (50 microliters) were further added to the resultant. The resultant was incubated at 4 degrees C. for 30 minutes, diluted in a measuring flask up to 50 mL with a staining medium (SM) having a temperature of 4 degrees C. and mixed by inversion, and centrifuged at 1,500 rpm at 4 degrees C. for 10 minutes. Then, the supernatant was removed. AUTOMACS (registered trademark) RUNNING BUFFER (obtained from Miltenyi Biotec GmbH) was added to the obtained precipitate by 1 mL per 5×10⁸ mononucleosis cells at the maximum. CD34-positive cells were separated from the resultant with a magnetic beads cell sorter (AUTOMACS (registered trademark), obtained from Miltenyi Biotec GmbH) using an LS column (obtained from Miltenyi Biotec GmbH).

The number of CD34-positive cells separated was 1.16×10⁷ cells, and the number of CD34-negative cells (mononucleosis) was 2.31×10⁸ cells.

### Test Example 1-1: Surface Marker Analysis 1

The relationship between the Young's modulus of the culture section and proliferation of hematopoietic stem cells was confirmed by the method described below.

### <Setting of Devices for Cell Culture>

The devices for cell culture of Example 1 and Comparative Example 1 were set in wells of a 96-well plate (formed of polystyrene, with a flat bottom, obtained from TPP Co., Ltd.). Wells in which no device for cell culture was set were used as control. All of these wells were run in triplicate (n=3).

### <Surface Coating of Devices for Cell Culture>

Fibronectin (obtained from SouthernBiotech Inc.) was diluted to 25 micrograms/mL with PBS(−) (obtained from Sigma-Aldrich Co. LLC), to prepare a fibronectin solution. The fibronectin solution was dropped by 50 microliters/well onto the culture sections of the devices for cell culture in the wells and onto the bottom surfaces of the control wells, and subsequently left to stand still at 37 degrees C. for 1 hour, to coat the surfaces of the culture sections and the bottom surfaces of the control wells with fibronectin. Next, the fibronectin solution was removed from the wells and the wells were washed once with PBS.

### <Culturing of Umbilical Cord Blood-Derived CD34-Positive Cells>

Ten percent by volume bovine serum albumin (BSA, obtained from Nacalai Tesque, Inc.) (2 mL), a 100 ng/microliter stem cell factor (human, obtained from SHENANDOAH, Inc.) (20 microliters), 10 ng/microliter thrombopoietin (human, obtained from SHENANDOAH, Inc.) (200 microliters), and 100 ng/microliter Flt-3 ligand (human, obtained from SHENANDOAH, Inc.) (20 microliters) were added to a serum-free medium (X-VIVO 10, obtained from Lonza Inc.) (18 mL), to prepare a culture liquid.

The umbilical cord blood-derived CD34-positive cells obtained in Preparation example 1 were seeded by 1,000 cells/well into the devices for cell culture and control wells that were surface-coated, and the culture liquid was added thereto by 200 microliters/well. Subsequently, the CD34-positive cells were statically cultured at a CO₂ concentration of 5% by volume at 37 degrees C. for 7 days.

### <Surface Marker Analysis>

### Preparation of Antibody Solution

Antibodies CD34-APC/Cy7 (obtained from BioLegend, Inc.) (0.7 microliters), CD38-PE/Cy7 (obtained from BioLegend, Inc.) (0.7 microliters), CD45RA-Brilliant Violet421 (obtained from BioLegend, Inc.) (0.7 microliters), and CD90-APC (obtained from BioLegend, Inc.) (1.1 microliters) were added to PBS (46.8 microliters) containing 2% by volume fetal bovine serum (FCS, obtained from Sigma-Aldrich Co. LLC), to prepare an antibody solution.

### Confirmation of [CD34+, CD90+, CD45RA− Cells]

The numbers of hematopoietic stem cells in the umbilical cord blood-derived CD34-positive cells before and after culture were confirmed by the method described below using surface markers for hematopoietic stem cells.

The culture liquid (200 microliters) containing the umbilical cord blood-derived CD34-positive cells obtained in Preparation example 1 (the cells before cultured) was transferred to tubes (n=3).

Cells on the devices for cell culture, which were obtained from culturing the umbilical cord blood-derived CD34-positive cells for 7 days, were floated in the culture liquid by sufficient pipetting in the wells. The whole amounts of the cell suspensions obtained in the respective wells were transferred to tubes.

The antibody solution described above was added by 10 microliters/tube to the tubes containing the cells before cultured and the tubes containing the cells after cultured, and then the cells were cultured in a shaded environment at 4 degrees C. for 30 minutes. Next, the tubes were centrifuged at 600 g for 10 minutes, and the supernatants were removed. Subsequently PBS (250 microliters) containing 2% by volume FCS, and flow cytometry cell counting beads (COUNTBRIGHT™ ABSOLUTE COUNTING BEADS, obtained from Thermo Fisher Scientific, Inc.) (5 microliters) were added to the tubes, to count the numbers of cells that expressed any of the antibodies with a flow cytometer (FACSCANTOII, obtained from Becton, Dickinson and Company).

Cells [CD34+, CD90+, CD45RA− cells] that were positive with CD34, positive with CD90, and negative with CD45RA were hematopoietic stem cells.

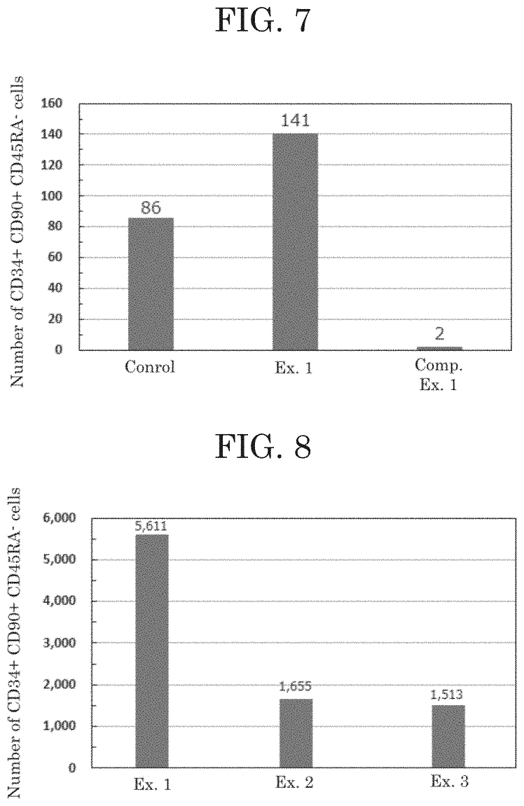

FIG. 7 indicates the results of analyzing the surface markers of the cells after cultured. The vertical axis represents the average number of cells of the hematopoietic stem cell fraction [CD34+, CD90+, CD45RA− cells] per well. The average number of cells of the hematopoietic stem cell fraction [CD34+, CD90+, CD45RA− cells] per well before culture was seven cells, although not indicated in FIG. 7.

From the result of FIG. 7, it was revealed that the number of hematopoietic stem cells when the device for cell culture of Example 1 having Young's modulus of 3 GPa at the culture section was used was significantly higher compared with the control in which no device for cell culture was used. On the other hand, the number of hematopoietic stem cells when the device for cell culture of Comparative Example 1 having Young's modulus of less than 3 GPa at the culture section was used was lower than the control.

It was inferred that closeness of the device for cell culture of Example 1 having Young's modulus of 3 GPa at the culture section to the hard environment of the cancellous bone in the bone marrow was one factor that brought about this result.

### Test Example 1-2: Surface Marker Analysis 2

The relationship between the average length (La) of the openings of the pores and proliferation of hematopoietic stem cells was confirmed in the same manners as in Test Example 1-1 in terms of <Surface coating of devices for cell culture> and <Surface marker analysis>, but in the below-described manners different from Test Example 1-1 in terms of <Setting of devices for cell culture> and <Culturing of umbilical cord blood-derived CD34-positive cells>.

### <Setting of Devices for Cell Culture>

The devices for cell culture of Example 1, Example 2, and Example 3 were set in wells of a 96-well plate (formed of polystyrene, with a flat bottom, obtained from TPP Co., Ltd.). Wells in which no device for cell culture was set were used as control. All of these wells were run in triplicate (n=3).

### <Culturing of Umbilical Cord Blood-Derived CD34-Positive Cells>

The umbilical cord blood-derived CD34-positive cells obtained in Preparation example 1 were seeded by 5,000 cells/well into the devices for cell culture and control wells that were surface-coated, and the culture liquid prepared in Test Example 1-1 was added thereto by 200 microliters/well. Subsequently, the CD34-positive cells were statically cultured at a CO₂ concentration of 5% by volume at 37 degrees C. for 7 days.

After seven days of culturing, the cells cultured in the devices for cell culture of Example 1, Example 2, and Example 3 were observed with a phase-contrast microscope, and then subjected to <Surface marker analysis> in the same manner as in Test Example 1-1.

FIG. 8 indicates the result of analyzing the surface markers. The vertical axis represents the average number of cells of the hematopoietic stem cell fraction [CD34+, CD90+, CD45RA− cells] per well. The average number of cells of the hematopoietic stem cell fraction [CD34+, CD90+, CD45RA− cells] per well before culture was 37 cells, although not indicated in FIG. 8.

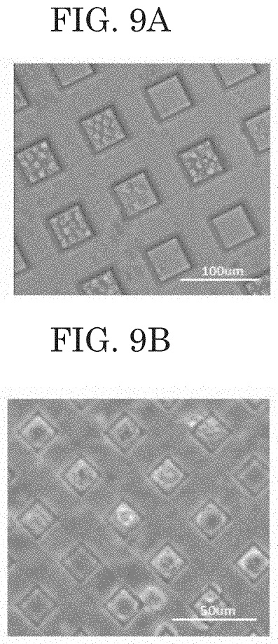

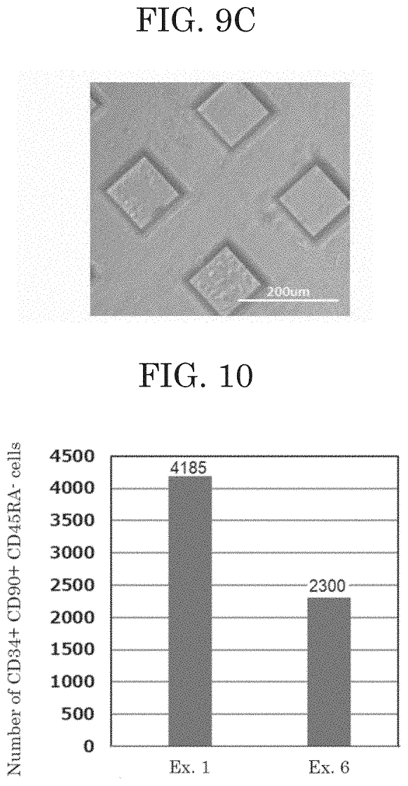

FIG. 9A to FIG. 9C are phase-contrast microscope images of the openings of the pores after seven days of culturing. In FIG. 9A to FIG. 9C, the regions surrounded by quadrangles are the pores in the surface of the device for cell culture, and it was confirmed that the cells had entered the pores.

Hematopoietic stem cells have a cell size of about from 10 micrometers through 15 micrometers. Therefore, only about one cell per pore was confirmed in the pores having an average length (La) of the openings of 15 micrometers (Example 2). A plurality of cells per pore were confirmed in the pores having an average length (La) of the openings of 100 micrometers (Example 3), but there were relatively wide gaps between the cells. On the other hand, when the pores having an average length (La) of the openings of 47 micrometers (Example 1) were closely observed, cells having substantially no gaps between the cells were confirmed per pore.

### Test Example 1-3: Surface Marker Analysis 3

The relationship between the Young's modulus of the culture section and proliferation of hematopoietic stem cells was further confirmed in the same manners as in Test Example 1-1 in terms of <Surface coating of devices for cell culture> and <Surface marker analysis>, but in the below-described manners different from Test Example 1-1 in terms of <Setting of devices for cell culture> and <Culturing of umbilical cord blood-derived CD34-positive cells>.

### <Setting of Devices for Cell Culture>

The devices for cell culture of Example 1 and Example 6 were set in wells of a 96-well plate (formed of polystyrene, with a flat bottom, obtained from TPP Co., Ltd.). All of these wells were run in duplicate (n=2).

### <Culturing of Umbilical Cord Blood-Derived CD34-Positive Cells>

The umbilical cord blood-derived CD34-positive cells obtained in Preparation example 1 were seeded by 5,000 cells/well into the devices for cell culture and control wells that were surface-coated, and the culture liquid prepared in Test Example 1-1 was added thereto by 200 microliters/well. Subsequently, the CD34-positive cells were statically cultured at a CO₂ concentration of 5% by volume at 37 degrees C. for 7 days.

After seven days of culturing, the cells cultured in the devices for cell culture of Example 1 and Example 6 were subjected to <Surface marker analysis> in the same manner as in Test Example 1-1.

FIG. 10 indicates the result of analyzing the surface markers of the cells after culture. The vertical axis represents the average number of cells of the hematopoietic stem cell fraction [CD34+, CD90+, CD45RA− cells] per well.

### Test Example 1-4: Surface Marker Analysis 4

The relationship between the average length [La] of the openings of the pores and average depth [H] of the pores, and proliferation of hematopoietic stem cells when the pores had approximately the same aspect ratio [H/La] was confirmed in the same manners as in Test Example 1-1 in terms of <Surface coating of devices for cell culture> and <Surface marker analysis>, but in the below-described manners different from Test Example 1-1 in terms of <Setting of devices for cell culture> and <Culturing of umbilical cord blood-derived CD34-positive cells>.

### <Setting of Devices for Cell Culture>

The devices for cell culture of Example 1, Example 2, Example 4, and Example 5 were set in wells of a 96-well plate (formed of polystyrene, with a flat bottom, obtained from TPP Co., Ltd.). All of these wells were run in duplicate (n=2).

### <Culturing of Umbilical Cord Blood-Derived CD34-Positive Cells>

The umbilical cord blood-derived CD34-positive cells obtained in Preparation example 1 were seeded by 5,000 cells/well into the devices for cell culture and control wells that were surface-coated, and the culture liquid prepared in Test Example 1-1 was added thereto by 200 microliters/well. Subsequently, the CD34-positive cells were statically cultured at a CO₂ concentration of 5% by volume at 37 degrees C. for 7 days.

After seven days of culturing, the cells cultured in the devices for cell culture of Example 1, Example 2, Example 4, and Example 5 were subjected to <Surface marker analysis> in the same manner as in Test Example 1-1.

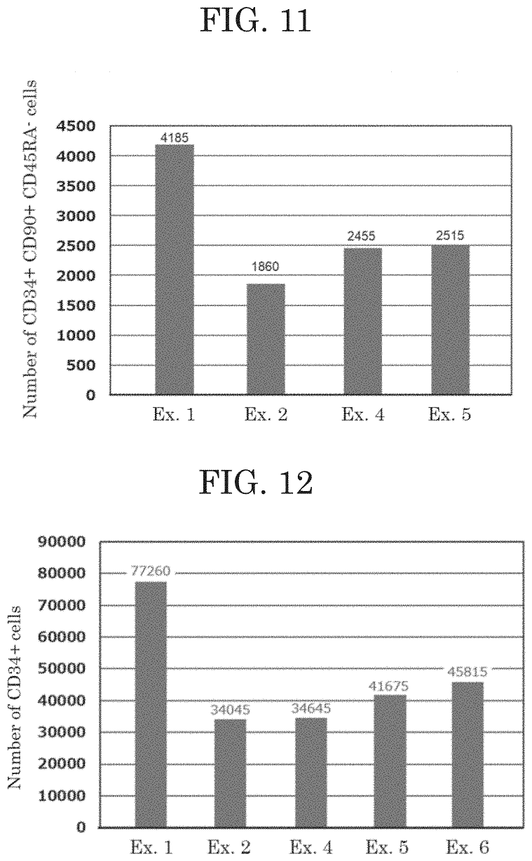

FIG. 11 indicates the result of analyzing the surface markers of the cells after culture. The vertical axis represents the average number of cells of the hematopoietic stem cell fraction [CD34+, CD90+, CD45RA− cells] per well.

### Test Example 1-5: Surface Marker Analysis 5

It was confirmed that hematopoietic progenitor cells were also included among cells proliferated on the devices for cell culture in addition to hematopoietic stem cells in the same manners as in Test Example 1-1 in terms of <Surface coating of devices for cell culture> and <Surface marker analysis>, but in the below-described manners different from Test Example 1-1 in terms of <Setting of devices for cell culture> and <Culturing of umbilical cord blood-derived CD34-positive cells>.

The method for <Surface marker analysis> was the same as the method used in Test Example 1-1, whereas human-derived hematopoietic stem cells and hematopoietic progenitor cells were cells that were CD34-positive [CD34+ cells].

### <Setting of Devices for Cell Culture>

The devices for cell culture of Example 1, Example 2, Example 4, Example 5, and Example 6 were set in wells of a 96-well plate (formed of polystyrene, with a flat bottom, obtained from TPP Co., Ltd.). All of these wells were run in duplicate (n=2).

### <Culturing of Umbilical Cord Blood-Derived CD34-Positive Cells>

The umbilical cord blood-derived CD34-positive cells obtained in Preparation example 1 were seeded by 5,000 cells/well into the devices for cell culture and control wells that were surface-coated, and the culture liquid prepared in Test Example 1-1 was added thereto by 200 microliters/well. Subsequently, the CD34-positive cells were statically cultured at a CO₂ concentration of 5% by volume at 37 degrees C. for 7 days.

After seven days of culturing, the cells cultured in the devices for cell culture of Example 1, Example 2, Example 4, Example 5, and Example 6 were subjected to <Surface marker analysis> in the same manner as in Test Example 1-1.

FIG. 12 indicates the result of analyzing the surface markers of the cells after culture. The vertical axis represents the average number of cells of the fraction including hematopoietic stem cells and hematopoietic progenitor cells [CD34+ cells] per well.

### Test Example 2: Methylcellulose Colony Assay

The self-replication ability and the multipotency of the hematopoietic stem cells and hematopoietic progenitor cells proliferated on the devices for cell culture were confirmed in the manner described below.

Using the devices for cell culture of Examples 1 to 3, <Setting of devices for cell culture>, <Surface coating of devices for cell culture>, and <Culturing of umbilical cord blood-derived CD34-positive cells> were performed in the same manners as in Test Example 1-1.

After the umbilical cord blood-derived CD34-positive cells were cultured for seven days, the cells in the devices for cell culture were floated in the culture liquid by sufficient pipetting in the wells. The whole amounts of the cell suspensions obtained in the respective wells were transferred to tubes. The tubes were centrifuged at 600 g for 10 minutes, and the supernatants were removed. Subsequently, an Iscove's Modified Dulbecco's Medium (IMDM, obtained from GE Healthcare Inc.) was added by 150 microliters/tube to the tubes to suspend the cells, to obtain cell suspensions. A MethoCult medium for measuring human hematopoietic progenitor cell colonies (obtained from STEMCELL Technologies, Inc.) (1.35 mL) was dispensed into petri dishes (formed of polystyrene, with a flat bottom, obtained from TPP Co., Ltd.), and the cell suspensions (150 microliters) were seeded into the petri dishes and statically cultured at a CO₂ concentration of 5% by volume at 37 degrees C. for 14 days. After the cells were cultured for 14 days, the colonies formed were observed with a phase-contrast microscope, to count the number of colonies of granulocytic lineage and monocytic lineage progenitor cells, the number of colonies of burst-forming unit-erythroid, and the number of mixed colonies in which blood cells of a plurality of lineages were mixed (STEMCELL Technologies. Inc., TECHNICAL MANUAL, Human Colony-Forming Unit (CFU) Assays Using MethoCult™, DOCUMENT #28404, VERSION 4.6.0, March 2019, see pp. 27-32).

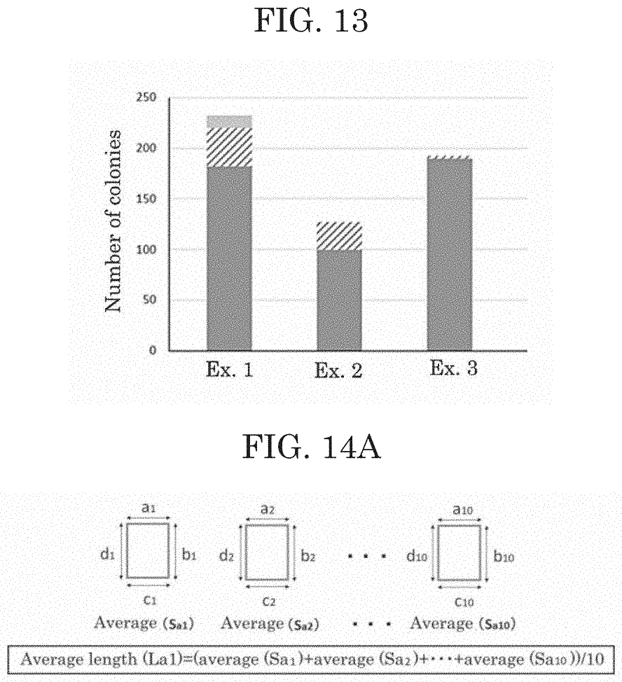

FIG. 13 indicates the result of the methylcellulose colony assay. The vertical axis represents the number of colonies.

From the result of FIG. 13, it was revealed that the devices for cell culture of Examples 1 to 3 succeeded in proliferating hematopoietic stem cells and hematopoietic progenitor cells. Above all, particularly when the device for cell culture of Example 1 was used, good proliferation of hematopoietic stem cells and hematopoietic progenitor cells was confirmed.

### INDUSTRIAL APPLICABILITY

Because the device for cell culture of the present invention can be produced easily at low costs and can efficiently proliferate hematopoietic stem cells or hematopoietic progenitor cells, or both the hematopoietic stem cells and the hematopoietic progenitor cells in vitro while maintaining the self-replication ability and the multipotency thereof, the device for cell culture can be suitably used as a scaffold for culturing and proliferating hematopoietic stem cells or hematopoietic progenitor cells, or both the hematopoietic stem cells and the hematopoietic progenitor cells in vitro. Moreover, the device for cell culture can be suitably used for, for example, studies about, for example, maintenance, proliferation, and differentiation of hematopoietic stem cells.

Because the cell culturing method of the present invention can efficiently proliferate hematopoietic stem cells or hematopoietic progenitor cells, or both the hematopoietic stem cells and the hematopoietic progenitor cells in vitro while maintaining the self-replication ability and the multipotency thereof, the cell culturing method can be suitably used for culturing hematopoietic stem cells or hematopoietic progenitor cells, or both the hematopoietic stem cells and the hematopoietic progenitor cells in vitro.

What is claimed is:

1. A device for cell culture, the device comprising:
a base material including a culture section used for culturing hematopoietic stem cells or hematopoietic progenitor cells, or both the hematopoietic stem cells and the hematopoietic progenitor cells,
wherein the culture section includes a plurality of pores, and
wherein a Young's modulus of the culture section measured according to JIS K 7161-1 and JIS K 7161-2 is at least 3 GPa.

2. The device for cell culture according to claim 1,
wherein the culture section is formed of polystyrene.

3. The device for cell culture according to claim 1,
wherein a ratio [H/La] of an average depth (H) of the pores to an average length (La) of openings of the pores is 1.1 or greater.

4. The device for cell culture according to claim 1,
wherein an average length of openings of the pores is from 30 micrometers through 80 micrometers.

5. The device for cell culture according to claim 1,
wherein an average depth of the pores is from 30 micrometers through 160 micrometers.

6. A cell culturing method, comprising:
culturing hematopoietic stem cells or hematopoietic progenitor cells, or both the hematopoietic stem cells and the hematopoietic progenitor cells using a device for cell culture,
wherein the device for cell culture comprises a base material including a culture section used for culturing the hematopoietic stem cells or the hematopoietic progenitor cells, or both the hematopoietic stem cells and the hematopoietic progenitor cells,
wherein the culture section includes a plurality of pores, and
wherein a Young's modulus of the culture section measured according to JIS K 7161-1 and JIS K 7161-2 is at least 3 GPa.
